(19) United States
(12) Patent Application Publication
(10) Pub. No.: US 2025/0157629 A1
(43) Pub. Date: May 15, 2025

(54) MEDICAL SUPPORT DEVICE, MEDICAL SUPPORT SYSTEM, MEDICAL SUPPORT METHOD, AND PROGRAM

(71) Applicant: FUJIFILM CORPORATION, Tokyo (JP)

(72) Inventor: Seiya TAKENOUCHI, Kanagawa (JP)

(21) Appl. No.: 18/915,369

(22) Filed: Oct. 15, 2024

(30) Foreign Application Priority Data
Nov. 10, 2023 (JP) 2023-192412

(51) Int. Cl. G16H 30/20; G16H 30/40

(52) U.S. Cl. CPC G16H 30/20; G16H 30/40

(57) ABSTRACT

A medical support device includes a processor. The processor acquires a plurality of medical images corresponding to a plurality of medical examinations, acquires a plurality of pieces of medical examination information that correspond to the plurality of medical examinations and that are each associated with the corresponding medical examination, and causes a processing device to execute recognition processing on each of the plurality of medical images in an order determined based on the plurality of pieces of medical examination information.

### CROSS-REFERENCE TO RELATED APPLICATIONS

This application claims priority under 35 USC 119 from Japanese Patent Application No. 2023-192412, filed on Nov. 10, 2023, the disclosure of which is incorporated by reference herein.

### BACKGROUND

### 1. Technical Field

The present disclosure relates to a medical support device, a medical support system, a medical support method, and a program.

### 2. Related Art

JP2023-046376A discloses a control method for controlling processing of data acquired from a medical imaging modality by using a plurality of data processors connected to a plurality of medical imaging modalities via a computer network. The control method disclosed in JP2023-046376A includes acquiring imaging information for imaging executed by one medical imaging modality among a plurality of medical imaging modalities, acquiring load information of the plurality of data processors before the imaging is completed, assigning, based on the acquired load information, at least a part of the plurality of data processors to processing of the data acquired by imaging based on the imaging information, and executing the processing of the acquired data via the assigned data processor.

WO2005/003977A discloses an image database system including a storage unit that accumulates medical image data, a control unit that manages the storage unit, an image database server that stores attribute information including key information on the medical image data accumulated in the storage unit and that executes relay-processing of the medical image data with an externally connected device, and a DICOM gateway that executes relay-processing of the medical image data with a plurality of modality devices externally connected to the image database server via a DICOM protocol, in which the storage unit, the control unit, the image database server, and the DICOM gateway are network-connected to each other via a switch. The image database system disclosed in WO2005/003977A is provided with a control unit group, an image database server group, and a DICOM gateway group by comprising at least a plurality of control units, a plurality of image database servers, and a plurality of DICOM gateways, separately, and is provided with a load balancer that executes load distribution control for each group based on header information of a request.

### SUMMARY

One embodiment according to the present disclosure provides a medical support device, a medical support system, a medical support method, and a program that can suppress, with high accuracy, a problem that occurs in a case in which recognition processing is executed on each of a plurality of medical images corresponding to a plurality of medical examinations.

A first aspect according to the present disclosure relates to a medical support device comprising: a processor, in which the processor acquires a plurality of medical images corresponding to a plurality of medical examinations, acquires a plurality of pieces of medical examination information that correspond to the plurality of medical examinations and that are each associated with the corresponding medical examination, and causes a processing device to execute recognition processing on each of the plurality of medical images in an order determined based on the plurality of pieces of medical examination information.

A second aspect according to the present disclosure relates to the medical support device according to the first aspect, in which the medical examination information includes middle-of-examination information clarified within a period during which the corresponding medical examination is performed.

A third aspect according to the present disclosure relates to the medical support device according to the first or second aspect, in which the medical examination information includes pre-examination information clarified before a period during which the corresponding medical examination is performed.

A fourth aspect according to the present disclosure relates to the medical support device according to any one of the first to third aspects, in which the medical examination information includes subject information on a subject who undergoes the corresponding medical examination.

A fifth aspect according to the present disclosure relates to the medical support device according to any one of the first to fourth aspects, in which the medical examination information includes operator information on an operator who performs the corresponding medical examination.

A sixth aspect according to the present disclosure relates to the medical support device according to any one of the first to fifth aspects, in which the medical image is obtained in the corresponding medical examination, and the medical examination information includes modality information on a modality used for imaging to obtain the medical image in the corresponding medical examination.

A seventh aspect according to the present disclosure relates to the medical support device according to any one of the first to sixth aspects, in which the medical image is obtained in the corresponding medical examination, the medical examination information includes an intermediate result of the corresponding medical examination, and the intermediate result is information specified based on the medical image obtained in the corresponding medical examination.

An eighth aspect according to the present disclosure relates to the medical support device according to the seventh aspect, in which the intermediate result includes feature region information that is information on a first feature region shown in the medical image.

A ninth aspect according to the present disclosure relates to the medical support device according to the eighth aspect, in which the first feature region is a lesion, and the intermediate result is a screening result of the lesion or a discrimination result of the lesion.

A tenth aspect according to the present disclosure relates to the medical support device according to any one of the first to ninth aspects, in which the processor transmits medical support information including an execution result obtained by executing the recognition processing to a device used for the medical examination corresponding to the medical image that is a target of the recognition processing executed to obtain the execution result included in the medical support information.

An eleventh aspect according to the present disclosure relates to the medical support device according to the tenth aspect, in which the medical support information includes information in which the medical image that is the target of the recognition processing executed to obtain the execution result included in the medical support information and the execution result are associated with each other.

A twelfth aspect according to the present disclosure relates to the medical support device according to the tenth or eleventh aspect, in which the processor transmits corresponding medical support information to the device in an order in which the execution result is obtained via the recognition processing executed on each of the plurality of medical images in the order.

A thirteenth aspect according to the present disclosure relates to the medical support device according to any one of the first to twelfth aspects, in which the recognition processing is processing of generating information on a second feature region by inputting the medical image to a trained model that receives input of an image showing a first region corresponding to the second feature region to generate information on the first region.

A fourteenth aspect according to the present disclosure relates to the medical support device according to any one of the first to thirteenth aspects, in which each of the plurality of medical examinations is an endoscopy, and each of the plurality of medical images is an endoscopic image obtained in the endoscopy.

A fifteenth aspect according to the present disclosure relates to a medical support system comprising: the medical support device according to any one of the first to fourteenth aspects; and a communication device that transmits the medical image to the medical support device and that receives an execution result obtained by executing the recognition processing.

A sixteenth aspect according to the present disclosure relates to a medical support method comprising: acquiring a plurality of medical images corresponding to a plurality of medical examinations; acquiring a plurality of pieces of medical examination information that correspond to the plurality of medical examinations and that are each associated with the corresponding medical examination; and causing a processing device to execute recognition processing on each of the plurality of medical images in an order determined based on the plurality of pieces of medical examination information.

A seventeenth aspect according to the present disclosure relates to a program causing a computer to execute a process comprising: acquiring a plurality of medical images corresponding to a plurality of medical examinations; acquiring a plurality of pieces of medical examination information that correspond to the plurality of medical examinations and that are each associated with the corresponding medical examination; and causing a processing device to execute recognition processing on each of the plurality of medical images in an order determined based on the plurality of pieces of medical examination information.

### BRIEF DESCRIPTION OF THE DRAWINGS

Exemplary embodiments of the technology of the disclosure will be described in detail based on the following figures, wherein:

FIG. 1 is a schematic configuration diagram showing an example of an overall configuration of a medical support system;

FIG. 2 is a conceptual diagram showing an example of the overall configuration of the medical support system;

FIG. 3 is a block diagram showing an example of a hardware configuration of an electric system of the medical support system;

FIG. 4 is a block diagram showing an example of main functions of a processor included in a server and an example of information stored in a storage included in the server;

FIG. 5 is a conceptual diagram showing an example of processing contents in an endoscope device and the server;

FIG. 6 is a conceptual diagram showing an example of the processing content in the server and an example of contents displayed on a screen of a display device included in the endoscope device;

FIG. 7 is a conceptual diagram showing an example of a processing content executed by the processor included in the endoscope device in a case in which the endoscope device acquires server processing information from the server;

FIG. 8 is a conceptual diagram showing an example of a processing content for deciding an order in which a plurality of medical images (in the example shown in FIG. 8, a medical image in processing target information obtained from a processor of an endoscope device used in a first endoscopy and a medical image in processing target information obtained from a processor of an endoscope device used in a second endoscopy) are used in recognition processing, based on processing request information in metadata included in each of a plurality of pieces of processing target information (in the example shown in FIG. 8, the processing target information obtained from the processor of the endoscope device used for the first endoscopy and the processing target information obtained from the processor of the endoscope device used for the second endoscopy) corresponding to a plurality of endoscope devices;

FIG. 9 is a conceptual diagram showing an example of processing contents of a controller and a recognition unit of the server;

FIG. 10 is a conceptual diagram showing an example of an aspect in which server processing information including information in which a medical image and processing specification information are associated with each other is transmitted from the server to the endoscope device;

FIG. 11 is a flowchart showing an example of a flow of a medical support process executed by the processor included in the server;

FIG. 12 is a conceptual diagram showing an example of a processing content for deciding an order in which a plurality of medical images (in the example shown in FIG. 12, a medical image in processing target information obtained from a processor of an endoscope device used in a first endoscopy and a medical image in processing target information obtained from a processor of an endoscope device used in a second endoscopy) are used in recognition processing, based on subject information in metadata included in each of a plurality of pieces of processing target information (in the example shown in FIG. 12, the processing target information obtained from the processor of the endoscope device used for the first endoscopy and the processing target information obtained from the processor of the endoscope device used for the second endoscopy) corresponding to a plurality of endoscope devices;

FIG. 13 is a conceptual diagram showing an example of a processing content for deciding an order in which a plurality of medical images (in the example shown in FIG. 13, a medical image in processing target information obtained from a processor of an endoscope device used in a first endoscopy and a medical image in processing target information obtained from a processor of an endoscope device used in a second endoscopy) are used in recognition processing, based on operator information in metadata included in each of a plurality of pieces of processing target information (in the example shown in FIG. 13, the processing target information obtained from the processor of the endoscope device used for the first endoscopy and the processing target information obtained from the processor of the endoscope device used for the second endoscopy) corresponding to a plurality of endoscope devices;

FIG. 14 is a conceptual diagram showing an example of a processing content for deciding an order in which a plurality of medical images (in the example shown in FIG. 14, a medical image in processing target information obtained from a processor of an endoscope device used in a first endoscopy and a medical image in processing target information obtained from a processor of an endoscope device used in a second endoscopy) are used in recognition processing based on modality information in metadata included in each of a plurality of pieces of processing target information (in the example shown in FIG. 14, the processing target information obtained from the processor of the endoscope device used for the first endoscopy and the processing target information obtained from the processor of the endoscope device used for the second endoscopy) corresponding to a plurality of endoscope devices;

FIG. 15 is a conceptual diagram showing an example of a processing content for deciding an order in which a plurality of medical images (in the example shown in FIG. 15, a medical image in processing target information obtained from a processor of an endoscope device used in a first endoscopy and a medical image in processing target information obtained from a processor of an endoscope device used in a second endoscopy) are used in recognition processing based on a recognition result in metadata included in each of a plurality of pieces of processing target information (in the example shown in FIG. 15, the processing target information obtained from the processor of the endoscope device used for the first endoscopy and the processing target information obtained from the processor of the endoscope device used for the second endoscopy) corresponding to a plurality of endoscope devices;

FIG. 16 is a conceptual diagram showing an example of an aspect in which server processing information including information in which the medical image and a bounding box as one of the recognition results are associated with each other is transmitted from the server to the endoscope device; and

FIG. 17 is a schematic configuration diagram showing a modification example of the overall configuration of the medical support system.

### DETAILED DESCRIPTION

Hereinafter, examples of embodiments of a medical support device, a medical support system, a medical support method, and a program according to the present disclosure will be described with reference to the accompanying drawings.

First, the terms used in the following description will be described.

CPU is an abbreviation for “central processing unit”. GPU is an abbreviation for “graphics processing unit”. GPGPU is an abbreviation for “general-purpose computing on graphics processing units”. APU is an abbreviation for “accelerated processing unit”. TPU is an abbreviation for “tensor processing unit”. RAM is an abbreviation for “random-access memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. AI is an abbreviation for “artificial intelligence”. BLI is an abbreviation for “blue light imaging”. LCI is an abbreviation for “linked color imaging”. WLI is an abbreviation for “white light imaging”. I/F is an abbreviation for “interface”. SSL is an abbreviation for “sessile serrated lesion”. LAN is an abbreviation for “local area network”. WAN is an abbreviation for “wide area network”. 5G is an abbreviation for “5th generation mobile communication system”. IC is an abbreviation for “integrated circuit”. MRI is an abbreviation for “magnetic resonance imaging”. CT is an abbreviation for “computed tomography”.

In the following description, a processor with a reference numeral (hereinafter, simply referred to as “processor”) may be one physical or virtual operation device or a combination of a plurality of physical or virtual operation devices. Further, the processor may be one type of operation device or a combination of a plurality of types of operation devices. Examples of the operation device include a CPU, a GPU, a GPGPU, an APU, and a TPU.

In the following description, a memory with a reference numeral is a memory such as a RAM that temporarily stores information, and is used as a work memory by the processor.

In the following description, a storage with a reference numeral is one or more non-volatile storage devices that store various programs, various parameters, and the like. Examples of the non-volatile storage device include a flash memory, a magnetic disk, and a magnetic tape.

Examples of the storage also include a cloud storage.

In the following embodiment, an external I/F with a reference numeral controls the transmission and the reception of various types of information among a plurality of devices connected to each other. Examples of the external I/F include a USB interface. A communication I/F including a communication processor, an antenna, and the like may be applied to the external I/F. The communication I/F controls communication among a plurality of computers. Examples of a communication standard applied to the communication I/F include a wireless communication standard including 5G, Wi-Fi (registered trademark), or Bluetooth (registered trademark).

In the following embodiment, “A and/or B” is synonymous with “at least one of A or B”. That is, “A and/or B” may mean only A, only B, or a combination of A and B. In the present specification, the same concept as “A and/or B” also applies to a case in which three or more matters are expressed by association with “and/or”.

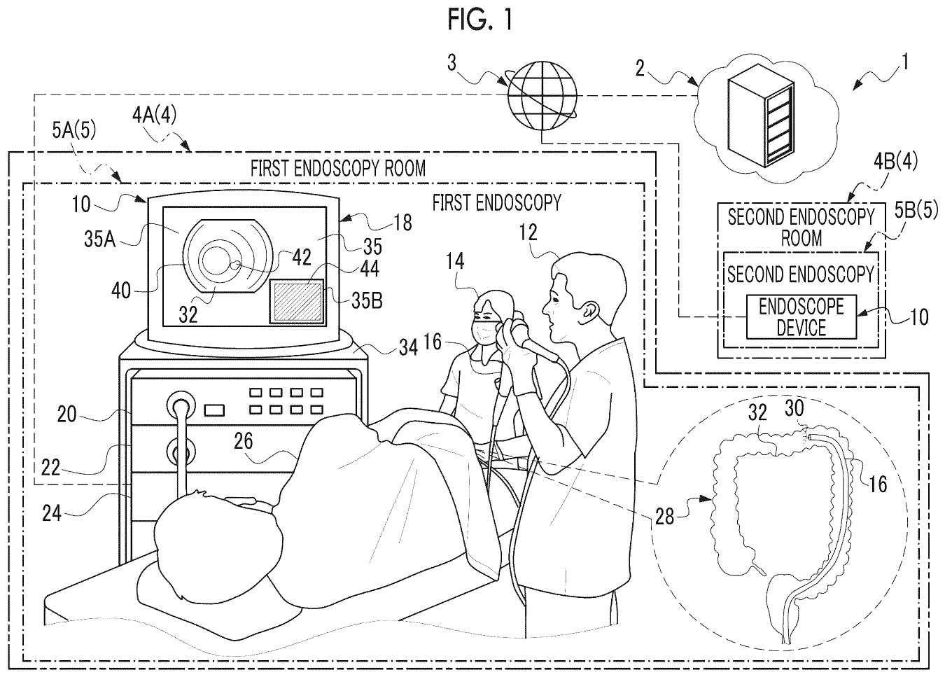

FIG. 1 is a schematic configuration diagram showing an example of an overall configuration of a medical support system 1. As shown in FIG. 1, the medical support system 1 comprises a server 2 and a plurality of endoscope devices 10, and the server 2 and the plurality of endoscope devices 10 are connected via a network 3 so as to be able to communicate with each other. Examples of the network 3 include the Internet. However, the Internet is merely an example, and examples of the network 3 also include a WAN and/or a LAN. In the present embodiment, the medical support system 1 is an example of a “medical support system” according to the present disclosure, the server 2 is an example of a “processing device” and a “medical support device” according to the present disclosure, and the endoscope device 10 is an example of a “device”, a “modality”, and a “communication device” according to the present disclosure.

The plurality of endoscope devices 10 are used by a plurality of doctors 12 in a plurality of endoscopies 5 performed in a plurality of endoscopy rooms 4. The endoscopy 5 is assisted by a staff member such as a nurse 14. In the example shown in FIG. 1, as examples of the plurality of endoscopy rooms 4, a first endoscopy room 4A and a second endoscopy room 4B are shown. In addition, in the example shown in FIG. 1, as examples of the plurality of endoscopies 5, a first endoscopy 5A and a second endoscopy 5B are shown. In the present embodiment, the endoscope device 10 is used by different doctors 12 in each of the first endoscopy 5A and the second endoscopy 5B. Here, a doctor 12 is an example of an “operator” according to the present disclosure. The first endoscopy 5A and the second endoscopy 5B are examples of a “plurality of medical examinations” according to the present disclosure. Each of the first endoscopy 5A and the second endoscopy 5B is an example of an “endoscopy” according to the present disclosure.

It should be noted that, in the present embodiment, in order to facilitate the understanding of the present disclosure, the first endoscopy room 4A and the second endoscopy room 4B are described as the plurality of endoscopy rooms 4, and the first endoscopy 5A and the second endoscopy 5B are described as the plurality of endoscopies 5, but this is merely an example, and the present disclosure is applicable even in a case in which there are three or more endoscopy rooms 4 and three or more endoscopies 5.

Information obtained by the endoscope device 10 is transmitted to the server 2 via the network 3. Examples of the server 2 include a cloud server. However, the cloud server is merely an example, and the server 2 may be an on-premises server or a personal computer. The server 2 receives the information transmitted from the endoscope device 10, executes processing using the received information, and transmits a processing result obtained by executing the processing to the endoscope device 10.

The endoscope device 10 comprises an endoscope 16, a display device 18, a light source device 20, a control device 22, and a function expansion device 24.

The endoscope device 10 is a modality applied to a subject 26 who undergoes the endoscopy 5. That is, the endoscope device 10 is a modality for performing a medical treatment on a large intestine 28 of the subject 26 (for example, a patient) by using the endoscope 16. In the present embodiment, the large intestine 28 is a target observed by the doctor 12. The subject 26 is an example of a “subject” according to the present disclosure.

The endoscope 16 is used by the doctor 12 and is inserted into a body of the subject 26. In the present embodiment, the endoscope 16 is inserted into the large intestine 28 that is a lumen organ of the subject 26. The endoscope device 10 causes the endoscope 16 inserted into the large intestine 28 of the subject 26 to image an inside of the large intestine 28 of the subject 26 and performs various medical treatments on the large intestine 28 as necessary. The endoscope device 10 acquires an image showing an aspect in the large intestine 28 by imaging the inside of the large intestine 28 of the subject 26, and outputs the image. In the present embodiment, the endoscope device 10 is an endoscope having an optical imaging function of capturing reflected light obtained by emitting light 30 inside the large intestine 28 and reflecting the light 30 from an intestinal wall 32 of the large intestine 28.

It should be noted that, although the endoscopy of the large intestine 28 is described as an example here, this is merely an example, and the present disclosure is applicable even in a case of an endoscopy of a lumen organ such as an upper gastrointestinal tract (for example, an esophagus, a stomach, and a duodenum) or a trachea.

The light source device 20, the control device 22, and the function expansion device 24 are installed on a wagon 34. The wagon 34 is provided with a plurality of stages along an up-down direction, and the function expansion device 24, the control device 22, and the light source device 20 are installed from a lower stage to an upper stage. In addition, the display device 18 is installed on the uppermost stage in the wagon 34.

The control device 22 controls the entire endoscope device 10. The function expansion device 24 is a device that expands functions of the endoscope device 10, and is retrofitted to the control device 22. The function expansion device 24 is operated in accordance with an instruction from the control device 22. The function expansion device 24 is connected to the server 2 via the network 3 so as to be able to communicate with each other and requests the provision of the service from the server 2 to receive the provision of the requested service from the server 2. The function expansion device 24 executes, in accordance with the instructions from the control device 22, various types of processing on the information obtained from the control device 22 and the information obtained from the server 2 (for example, an image obtained by imaging the intestinal wall 32 via the endoscope 16, and the processing result of the server 2).

The display device 18 displays various types of information including the image.

Examples of the display device 18 include a liquid-crystal display and an EL display. In addition, a tablet terminal with a display may be used instead of or together with the display device 18.

A screen 35 is displayed on the display device 18. The screen 35 includes a plurality of display regions. The plurality of display regions are arranged on the screen 35. In the example shown in FIG. 1, a first display region 35A and a second display region 35B are shown as examples of the plurality of display regions. In the example shown in FIG. 1, a first display region 35A and a second display region 35B are shown as examples of the plurality of display regions. A size of the first display region 35A is larger than a size of the second display region 35B. The first display region 35A is used as a main display region, and the second display region 35B is used as a sub-display region. A size relationship between the first display region 35A and the second display region 35B is not limited thereto, and need only be a size relationship that fits on the screen 35.

A medical image 40 is displayed in the first display region 35A. The medical image 40 is an endoscopic image obtained in the endoscopy 5, that is, an endoscopic image acquired by imaging, via the endoscope 16, the intestinal wall 32 in the large intestine 28 of the subject 26 during the endoscopy 5. The example shown in FIG. 1 shows an image in which the intestinal wall 32 is shown, as an example of the medical image 40.

The medical image 40 is displayed in the first display region 35A as a moving image or a still image. Here, the medical image 40 is an example of a “medical image” and an “endoscopic image” according to the present disclosure.

The intestinal wall 32 shown in the medical image 40 includes a lesion 42 (for example, in the example shown in FIG. 1, one lesion 42) as a region of interest (that is, an observation target region) focused on by the doctor 12, and the doctor 12 can visually recognize an aspect of the intestinal wall 32, including the lesion 42, through the medical image 40. In the present embodiment, the lesion 42 is an example of a “first feature region”, a “second feature region”, and a “lesion” according to the present disclosure.

There are various types of the lesion 42, and examples of the types of the lesion 42 include a neoplastic polyp and a non-neoplastic polyp. Examples of the type of the neoplastic polyp include an adenomatous polyp (for example, SSL). Examples of the type of the non-neoplastic polyp include a hamartomatous polyp, a hyperplastic polyp, and an inflammatory polyp. It should be noted that the types shown here are types assumed in advance as the types of the lesion 42 in a case in which the endoscopy is performed on the large intestine 28, and the types of the lesion 42 may be different depending on the organ on which the endoscopy is performed.

In the present embodiment, for convenience of description, the form example is described in which one lesion 42 is shown in the medical image 40, but the present disclosure is not limited thereto, and the present disclosure is applicable even in a case in which a plurality of lesions 42 are shown in the medical image 40.

In the present embodiment, the lesion 42 is shown, but this is merely an example, and the region of interest (that is, the observation target region) focused on by the doctor 12 may be a feature region having some unique feature, such as an organ (for example, a duodenal papilla), a mark, an artificial treatment tool (for example, an artificial clip), a treated region (for example, a region in which a trace of removal of a polyp or the like remains), or the like.

The image displayed in the first display region 35A is one medical image 40 included in the moving image including a plurality of medical images 40 in time series. That is, the plurality of medical images 40 in time series are displayed in the first display region 35A at a predetermined frame rate (for example, several tens of frames/second).

Examples of the moving image displayed in the first display region 35A include a moving image of a live-view method. The live-view method is merely an example, and a moving image which is temporarily stored in a memory or the like and then displayed, such as a moving image of a post-view method, may be used. In addition, each frame included in a recorded moving image stored in a memory or the like may be reproduced and displayed on the screen 35 (for example, the first display region 35A) as the medical image 40.

In the screen 35, the second display region 35B is adjacent to the first display region 35A and is displayed at the lower right in the screen 35 in front view. The display position of the second display region 35B may be any position as long as the display position is within the screen 35 of the display device 18, but it is preferable that the second display region 35B is displayed at a position comparable to the medical image 40.

In the second display region 35B, auxiliary information 44 for assisting the doctor 12 in making a medical determination or the like in the endoscopy is displayed. The auxiliary information 44 is information referred to by the doctor 12. Examples of the auxiliary information 44 include various types of information on the subject 26 into which the endoscope 16 is inserted and/or various types of information obtained by executing a medical support process described later.

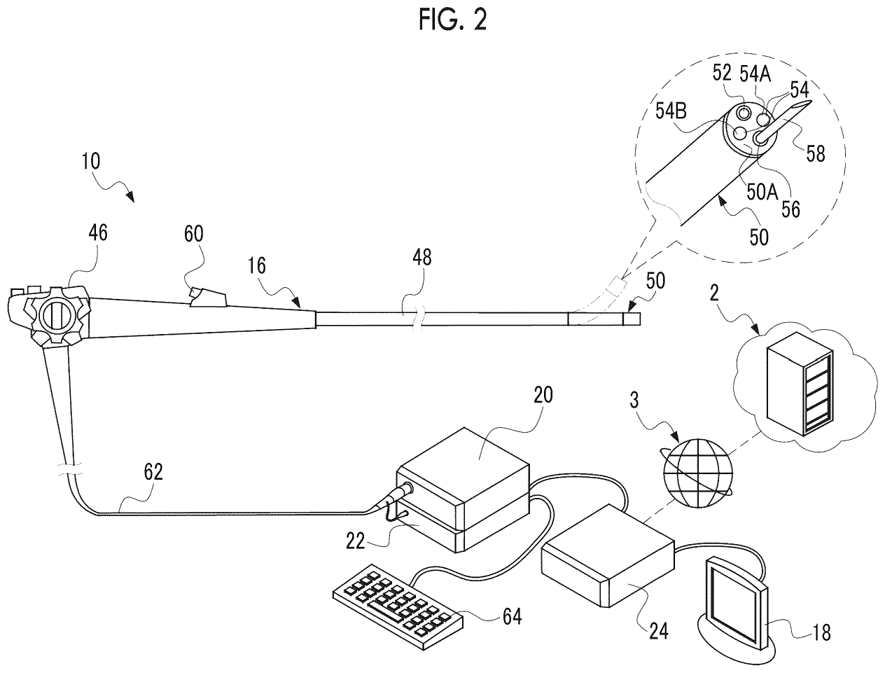

FIG. 2 is a conceptual diagram showing an example of an overall configuration of the endoscope device 10. As shown in FIG. 2, the endoscope 16 comprises an operating part 46 and an insertion part 48. The insertion part 48 is partially bent by operating the operating part 46. The insertion part 48 is inserted into the large intestine 28 while being bent in accordance with a shape of the large intestine 28 (see FIG. 1) in response to the operation performed on the operating part 46 by the doctor 12 (see FIG. 1).

A distal end part 50 of the insertion part 48 is provided with a camera 52, an illumination device 54, and a treatment tool opening 56. The camera 52 and the illumination device 54 are provided on a distal end surface 50A of the distal end part 50. It should be noted that, here, although the form example is described in which the camera 52 and the illumination device 54 are provided on the distal end surface 50A of the distal end part 50, this is merely an example, and the endoscope 16 may be configured as a side-view endoscope by providing the camera 52 and the illumination device 54 on a side surface of the distal end part 50.

The camera 52 is mounted in the endoscope 16, is inserted into a body cavity of the subject 26, and images the observation target region to generate the medical image 40 as an endoscopic image. In the present embodiment, the camera 52 generates the moving image including the plurality of medical images 40 in time series by imaging the inside of the body of the subject 26 (for example, the inside of the large intestine 28) in accordance with a predetermined frame rate. Examples of the camera 52 include a CMOS camera. However, this is merely an example, and another type of camera such as a CCD camera may be used.

The illumination device 54 includes illumination windows 54A and 54B. The illumination device 54 emits the light 30 (see FIG. 1) through the illumination windows 54A and 54B. Examples of the type of the light 30 emitted from the illumination device 54 include visible light (for example, white light) and invisible light (for example, near-infrared light). The illumination device 54 emits special light through the illumination windows 54A and 54B. Examples of the special light include light for BLI and/or light for LCI. The camera 52 images the inside of the large intestine 28 by using an optical method in a state in which the light 30 is emitted inside the large intestine 28 by the illumination device 54.

The treatment tool opening 56 is an opening for allowing a treatment tool 58 to protrude from the distal end part 50. In addition, the treatment tool opening 56 is also used as a suction port for suctioning blood, body waste, and the like and as a delivery port for sending out a fluid.

A treatment tool insertion port 60 is formed at the operating part 46, and the treatment tool 58 is inserted into the insertion part 48 through the treatment tool insertion port 60. The treatment tool 58 passes through the insertion part 48 and protrudes from the treatment tool opening 56 to the outside. In the example shown in FIG. 2, an aspect is shown in which a puncture needle as the treatment tool 58 protrudes from the treatment tool opening 56. Here, the puncture needle is shown as the treatment tool 58, but this is merely an example, and the treatment tool 58 may be a grasping forceps, a papillotomy knife, a snare, a catheter, a guide wire, a cannula, and/or a puncture needle with a guide sheath.

The endoscope 16 is connected to the light source device 20 and the control device 22 via a universal cord 62. The function expansion device 24 and a reception device 64 are connected to the control device 22. The display device 18 is also connected to the function expansion device 24 along with the server 2. That is, the control device 22 is connected to the server 2 and the display device 18 via the function expansion device 24.

Here, since the function expansion device 24 is shown as an external device for expanding the function executed by the control device 22, the form example is described in which the control device 22 and the display device 18 are indirectly connected to each other via the function expansion device 24, but this is merely an example. For example, the display device 18 may be directly connected to the control device 22. In this case, for example, a function of the function expansion device 24 need only be installed in the control device 22.

The reception device 64 receives an instruction from the doctor 12 and outputs the received instruction, as an electric signal, to the control device 22. Examples of the reception device 64 include a keyboard, a mouse, a touch panel, a foot switch, a microphone, and/or a remote operation device.

The control device 22 controls the light source device 20, controls the transmission and the reception of various signals with the camera 52, and controls the transmission and the reception of various signals with the function expansion device 24.

The light source device 20 emits light under the control of the control device 22 to supply the light to the illumination device 54. The illumination device 54 is provided with a built-in light guide, and the light supplied from the light source device 20 is emitted from the illumination windows 54A and 54B via the light guide. The light emitted from the illumination windows 54A and 54B is the light 30 (see FIG. 1). The control device 22 causes the camera 52 to perform the imaging, acquires the medical image 40 (see FIG. 1) from the camera 52, and outputs the acquired medical image 40 to a predetermined output destination (for example, the function expansion device 24).

The function expansion device 24 supports the medical treatment (here, as an example, the endoscopy) by executing various types of processing on the medical image 40 input from the control device 22. The function expansion device 24 outputs the medical image 40 subjected to various types of processing to a predetermined output destination (for example, the display device 18).

It should be noted that, here, the form example is described in which the medical image 40 output from the control device 22 is output to the display device 18 via the function expansion device 24, but this is merely an example. For example, an aspect may be adopted in which the control device 22 and the display device 18 are connected to each other, and the medical image 40 subjected to the image processing by the function expansion device 24 is displayed on the display device 18 via the control device 22.

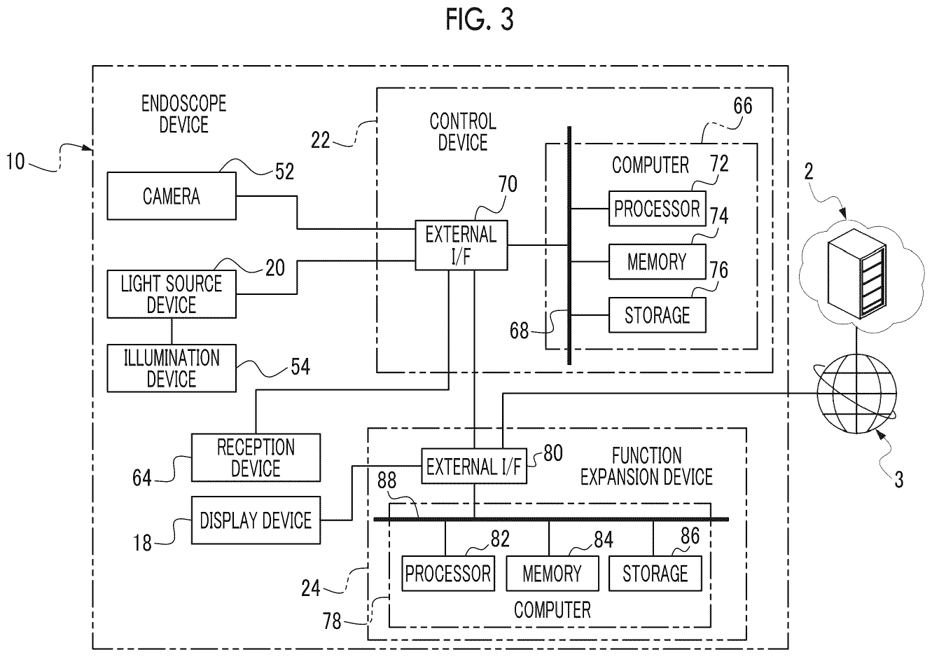

FIG. 3 is a block diagram showing an example of a hardware configuration of an electric system of the endoscope device 10. As shown in FIG. 3, the control device 22 comprises a computer 66 and an external I/F 70. The computer 66 comprises a processor 72, a memory 74, and a storage 76. The processor 72, the memory 74, the storage 76, and the external I/F 70 are connected to a bus 68. The processor 72 controls the entire control device 22. The memory 74 and the storage 76 are used by the processor 72.

The external I/F 70 controls the transmission and the reception of various types of information between one or more devices (hereinafter also referred to as “first external devices”) present outside the control device 22 and the processor 72.

The camera 52 is connected to the external I/F 70 as one of the first external devices, and the external I/F 70 controls the transmission and the reception of various types of information between the camera 52 and the processor 72. The processor 72 controls the camera 52 via the external I/F 70. In addition, the processor 72 acquires, via the external I/F 70, the medical image 40 (see FIG. 1) obtained by imaging the inside of the large intestine 28 (see FIG. 1) via the camera 52.

The light source device 20 is connected to the external I/F 70 as one of the first external devices, and the external I/F 70 controls the transmission and the reception of various types of information between the light source device 20 and the processor 72. The light source device 20 supplies the light to the illumination device 54 under the control of the processor 72. The illumination device 54 emits the light supplied from the light source device 20.

The reception device 64 is connected to the external I/F 70 as one of the first external devices, and the processor 72 acquires the instruction received by the reception device 64 via the external I/F 70 and executes processing in accordance with the acquired instruction.

The function expansion device 24 comprises a computer 78 and an external I/F 80. The computer 78 comprises a processor 82, a memory 84, and a storage 86. The processor 82, the memory 84, the storage 86, and the external I/F 80 are connected to a bus 88. It should be noted that, since a hardware configuration (that is, the processor 82, the memory 84, and the storage 86) of the computer 78 is basically the same as a hardware configuration of the computer 66, the description of the hardware configuration of the computer 78 will be omitted here.

The external I/F 80 controls the transmission and the reception of various types of information between one or more devices (hereinafter also referred to as “second external devices”) present outside the function expansion device 24 and the processor 82.

The control device 22 is connected to the external I/F 80 as one of the second external devices. In the example shown in FIG. 3, the external I/F 70 of the control device 22 is connected to the external I/F 80. The external I/F 80 controls the transmission and the reception of various types of information between the processor 82 of the function expansion device 24 and the processor 72 of the control device 22. For example, the processor 82 acquires the medical image 40 (see FIG. 1) from the processor 72 of the control device 22 via the external I/F 70 and the external I/F 80, and executes various types of processing on the acquired medical image 40.

The display device 18 is connected to the external I/F 80 as one of the second external devices. The processor 82 displays various types of information (for example, the medical image 40) on the display device 18 by controlling the display device 18 via the external I/F 80.

The server 2 is connected to the external I/F 80 as one of the second external devices via the network 3. The processor 82 executes the transmission and the reception of various types of information with the server 2 via the external I/F 80. For example, the external I/F 80 transmits the medical images 40 acquired from the camera 52 to the server 2 via the processor 82. The server 2 receives the medical images 40 transmitted from the external I/F 80, executes processing using at least one medical image 40 included in the received medical images 40, and transmits a processing result to the function expansion device 24. The external I/F 80 receives the processing result transmitted from the server 2. The processor 82 acquires the processing result received by the external I/F 80.

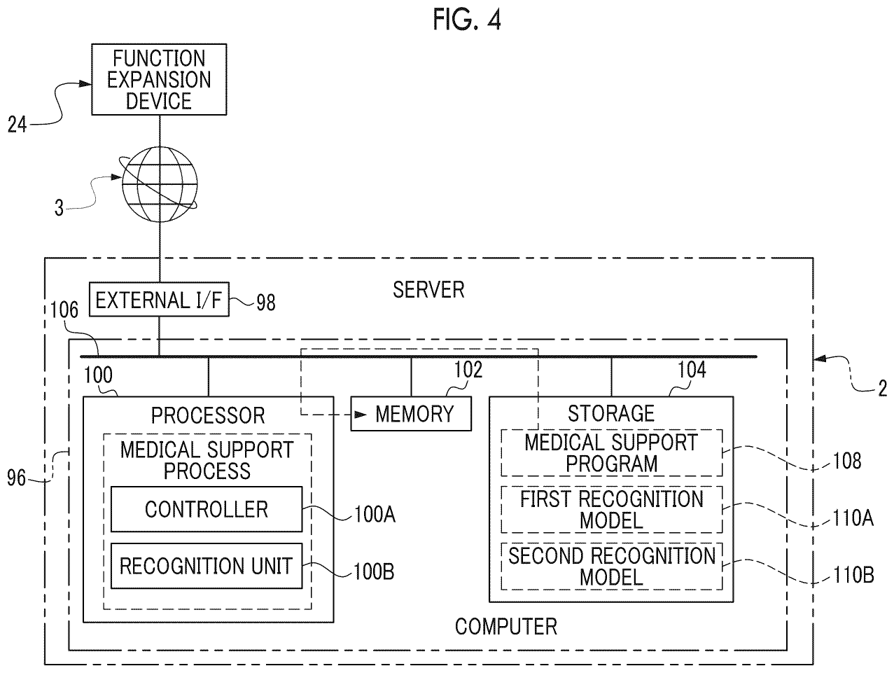

FIG. 4 is a block diagram showing an example of a hardware configuration of an electric system of the server 2. As shown in FIG. 4, the server 2 comprises a computer 96 and an external I/F 98. The computer 96 comprises a processor 100, a memory 102, and a storage 104. The processor 100, the memory 102, the storage 104, and the external I/F 98 are connected to a bus 106. Here, the computer 96 is an example of a “computer” according to the present disclosure, and the processor 100 is an example of a “processor” according to the present disclosure. It should be noted that, since a hardware configuration (that is, the processor 100, the memory 102, and the storage 104) of the computer 96 is basically the same as the hardware configuration of the computer 66, the description of the hardware configuration of the computer 96 will be omitted here.

The external I/F 98 controls the transmission and the reception of various types of information between one or more devices (hereinafter also referred to as “third external devices”) present outside the server 2 and the processor 100. The function expansion device 24 is connected to the external I/F 98 as one of the third external devices via the network 3. The processor 100 executes the transmission and the reception of various types of information with the function expansion device 24 via the external I/F 98. For example, the external I/F 98 receives various types of information such as the medical image 40 transmitted from the function expansion device 24. The processor 100 executes processing using the information received by the external I/F 98 and transmits a processing result to the function expansion device 24.

Meanwhile, in recent years, a technique has been developed of recognizing the lesion 42 by executing object recognition processing on the medical image 40 via a trained model that has been optimized by training a model (for example, a neural network) through machine learning, and displaying a recognition result of the object recognition processing or information based on the recognition result of the object recognition processing on the screen 35.

The object recognition processing executed on the medical image 40 has a very large processing load. Therefore, currently, a technology is being studied for reducing the processing load and traffic generated in a case in which the endoscope device 10 causes the server 2, which is communicably connected to the endoscope device 10 via the network 3, to execute the object recognition processing.

However, in a case in which the plurality of endoscope devices 10 share the server 2, it is assumed that the server 2 simultaneously executes the object recognition processing on the plurality of medical images 40 transmitted from the plurality of endoscope devices 10. In a case in which access to the server 2 via the plurality of endoscope devices 10 is concentrated, the processing load and traffic applied to the server 2 are extremely large, and, as a result, a time lag from a request for the object recognition processing made to the server 2 by each of the plurality of endoscope devices 10 to the reception of the processing result (that is, the processing result of the object recognition processing) of the server 2 is lengthened. In this case, for example, a discrepancy occurs between the content of the medical image 40 displayed on the screen 35 of each endoscope device 10 and the content of the processing result (for example, the content of the processing result irrelevant to the content of the medical image 40 is displayed on the screen 35). In addition, there is a risk that the server 2 may go down due to an overload on the server 2 caused by the object recognition processing being executed simultaneously on the plurality of medical images 40 transmitted from the plurality of endoscope devices 10. Meanwhile, it is necessary for the doctor 12 who is performing the endoscopy 5 with a high degree of urgency to understand the processing result of the server 2 earlier than the doctor 12 who is performing the endoscopy 5 with a low degree of urgency.

Therefore, in order to solve such a problem, in the present embodiment, as shown in FIG. 4 as an example, the medical support process is executed by the processor 100 of the server 2. A medical support program 108, a first recognition model 110A, and a second recognition model 110B are stored in the storage 104. The medical support program 108 is an example of a “program” according to the present disclosure. The first recognition model 110A and the second recognition model 110B are examples of a “trained model” according to the present disclosure.

The processor 100 reads out the medical support program 108 from the storage 104 and executes the readout medical support program 108 on the memory 102 to execute the medical support process. The medical support process is realized by the processor 100 operating as a controller 100A and a recognition unit 100B in accordance with the medical support program 108 executed on the memory 102. As will be described in more detail later, the first recognition model 110A and the second recognition model 110B are used by the recognition unit 100B.

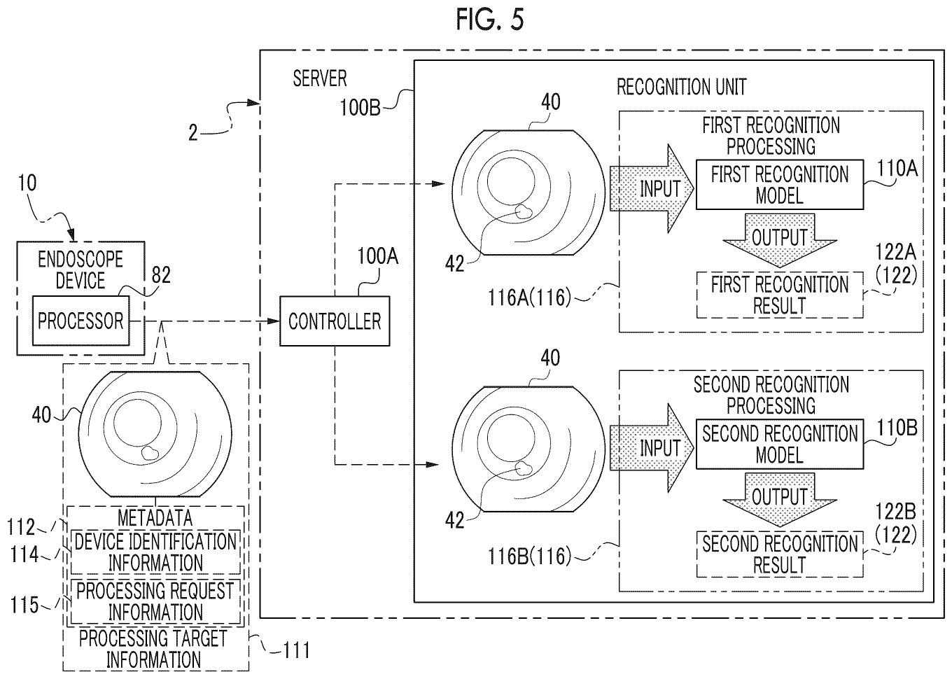

FIG. 5 is a conceptual diagram showing an example of the processing contents executed by the server 2 and the endoscope device 10. As shown in FIG. 5, the server 2 communicates with the endoscope device 10. The endoscope device 10 transmits processing target information 111 corresponding to the endoscopy 5 to the server 2. The processing target information 111 is an example of “medical examination information” according to the present disclosure.

The processing target information 111 is information that is a target processed by the server 2 and that is related to the corresponding endoscopy 5. In the server 2, the controller 100A acquires the processing target information 111 transmitted from the endoscope device 10 via the external I/F 98 (see FIG. 4). The medical image 40 and metadata 112 are included in the processing target information 111. The metadata 112 is data related to the medical image 40 and is associated with the medical image 40.

In the server 2, the controller 100A outputs the medical image 40 to the recognition unit 100B. The recognition unit 100B recognizes the feature region (for example, the lesion 42 shown in the medical image 40) in the medical image 40 based on the medical image 40 input from the controller 100A. In order to realize this, the recognition unit 100B executes recognition processing 116 on the medical image 40 input from the controller 100A. The recognition processing 116 is an example of “recognition processing” according to the present disclosure.

The recognition processing 116 is processing using AI (hereinafter also referred to as “AI processing”). Each time the medical image 40 is acquired by the server 2, the recognition processing 116 is executed on the acquired medical image 40. In the example shown in FIG. 5, as an example of the recognition processing 116, first recognition processing 116A and second recognition processing 116B are shown. The controller 100A causes the recognition unit 100B to selectively execute the first recognition processing 116A and the second recognition processing 116B. The first recognition processing 116A is processing of recognizing the lesion 42 shown in the medical image 40 (so-called screening), and the second recognition processing 116B is processing of discriminating the lesion 42 shown in the medical image 40 (that is, processing of specifying a specific type and/or subtype of the lesion 42).

The metadata 112 associated with the medical image 40 includes a plurality of types of information such as device identification information 114 and processing request information 115. The device identification information 114 is information for specifying the endoscope device 10. Examples of the device identification information 114 include an address for specifying a location of the endoscope device 10 on the network 3, and a model number of the endoscope device 10. For example, the device identification information 114 is referred to by the server 2 in a case in which the server 2 executes unique processing for each of the plurality of endoscope devices 10 (for example, in a case in which the recognition processing 116 is executed by the server 2 in response to a request from the endoscope device 10, and the processing result of the recognition processing 116 is transmitted to the endoscope device 10 that is a request source).

The processing request information 115 is information for requesting the recognition unit 100B to execute the recognition processing 116. The information for requesting the recognition unit 100B to execute the recognition processing 116 refers to, for example, information for requesting the recognition unit 100B to execute any one of the first recognition processing 116A or the second recognition processing 116B. The controller 100A causes the recognition unit 100B to selectively execute the first recognition processing 116A and the second recognition processing 116B in accordance with the processing request information 115 in the metadata 112 associated with the medical image 40 that is the processing target.

The first recognition model 110A is used for the first recognition processing 116A. The first recognition model 110A is a trained model for object recognition via a bounding box method using AI. The first recognition model 110A has been optimized by training a neural network through machine learning using first training data. That is, the first recognition model 110A is a trained model that has been optimized to receive input of first example data to generate first correct answer data.

The first training data is a data set including a plurality of data (that is, data for a plurality of frames) in which the first example data and the first correct answer data are associated with each other. The first example data is an image corresponding to the medical image 40 (in other words, an image assuming the medical image 40). A first example of the image corresponding to the medical image 40 is an image obtained by actually imaging the inside of the large intestine via the camera. A second example of the image assuming the medical image 40 is a virtually generated image (for example, an image generated by generative AI). The first correct answer data refers to correct answer data (that is, an annotation) for the first example data. Here, as an example of the first correct answer data, an annotation for specifying a position of the lesion shown in the image used as the first example data is used. Here, the lesion shown in the image used as the first example data is an example of a “first region” according to the present disclosure, the first example data is an example of an “image showing a first region” according to the present disclosure, and the first correct answer data is an example of “information on the first region” according to the present disclosure.

In a case in which the recognition unit 100B executes the first recognition processing 116A on the medical image 40 in accordance with the instruction from the controller 100A, the recognition unit 100B inputs the medical image 40, which is input from the controller 100A, to the first recognition model 110A. As a result, the first recognition model 110A recognizes the position of the lesion 42, which is shown in the input medical image 40, within the medical image 40, generates a first recognition result 122A that is a recognition result, and outputs the first recognition result 122A.

The second recognition model 110B is used for the second recognition processing 116B. The second recognition model 110B is a trained model for object recognition via a bounding box method using AI. The second recognition model 110B has been optimized by training a neural network through machine learning using second training data. That is, the second recognition model 110B is a trained model that has been optimized to receive input of second example data to generate second correct answer data.

The second training data is a data set including a plurality of data (that is, data for a plurality of frames) in which the second example data and the second correct answer data are associated with each other. A first example of the second example data is the same data as the first example data (that is, the image corresponding to medical image 40). A second example of the second example data is an image in which a region showing a lesion is cut out from the image corresponding to the medical image 40 by using a predetermined frame (for example, a circumscribing rectangular frame). A third example of the second example data is an image showing a lesion itself corresponding to the lesion 42. The second correct answer data refers to correct answer data (that is, an annotation) for the second example data. Here, as an example of the second correct answer data, an annotation is used for specifying the geometrical characteristics (for example, position, size, and shape) of the lesion 42 shown in the image used as the second example data, the type of lesion 42, and the subtype of lesion 42 (for example, a pedunculated type, a sub-pedunculated type, a sessile type, a surface protrusion type, a surface flat type, and a surface concave type).

In a case in which the recognition unit 100B executes the second recognition processing 116B on the medical image 40 in accordance with the instruction from the controller 100A, the recognition unit 100B inputs the medical image 40, which is input from the controller 100A, to the second recognition model 110B. As a result, the second recognition model 110B recognizes the geometrical characteristics of the lesion 42 shown in the input medical image 40, the type of the lesion 42, the subtype of the lesion 42, and the like, generates a second recognition result 122B that is a recognition result, and outputs the second recognition result 122B. It should be noted that, in the following description, in a case in which it is not necessary to distinguish between the first recognition result 122A and the second recognition result 122B, the first recognition result 122A and the second recognition result 122B are referred to as “recognition result 122”. In the present embodiment, the recognition result 122 is an example of an “execution result” and “information on a second feature region” according to the present disclosure.

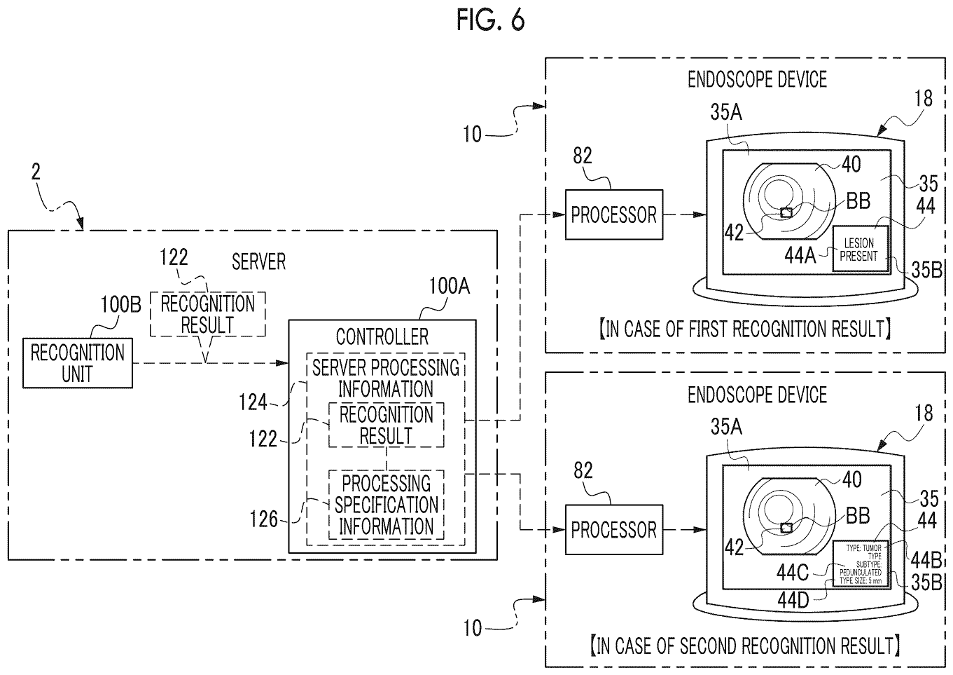

FIG. 6 is a conceptual diagram showing an example of the processing contents executed by the server 2 and the plurality of endoscope devices 10. As shown in FIG. 6, the controller 100A acquires the recognition result 122 from the recognition unit 100B. Then, the controller 100A generates server processing information 124. The server processing information 124 is an example of “medical support information” according to the present disclosure.

The server processing information 124 includes the recognition result 122 and processing specification information 126 acquired from the recognition unit 100B, and the recognition result 122 and the processing specification information 126 are associated with each other.

The processing specification information 126 is information for specifying the type of the recognition processing 116 corresponding to the recognition result 122, that is, information for specifying the type of the recognition processing 116 executed by the recognition unit 100B to obtain the recognition result 122 (that is, the first recognition processing 116A or the second recognition processing 116B). In a case in which the recognition result 122 acquired from the recognition unit 100B by the controller 100A is the first recognition result 122A, the controller 100A generates, as the processing specification information 126, information for specifying that the recognition processing 116 executed by the recognition unit 100B is the first recognition processing 116A. In a case in which the recognition result 122 acquired from the recognition unit 100B by the controller 100A is the second recognition result 122B, the controller 100A generates, as the processing specification information 126, information for specifying that the recognition processing 116 executed by the recognition unit 100B is the second recognition processing 116B.

The controller 100A transmits the server processing information 124 to the endoscope device 10, which is the request source that requests the server 2 to execute the recognition processing 116, by referring to the processing request information 115 (see FIG. 5), via the external I/F 98 (see FIG. 4). In the endoscope device 10, the processor 82 acquires the server processing information 124 transmitted from the server 2 via the external I/F 80 (see FIG. 3).

The processor 82 specifies whether the recognition processing 116 executed by the recognition unit 100B is the first recognition processing 116A or the second recognition processing 116B from the processing specification information 126 included in the server processing information 124 acquired via the external I/F 80 (see FIG. 3). In a case in which the recognition processing 116 executed by the recognition unit 100B is the first recognition processing 116A, the processor 82 displays information based on the first recognition result 122A included in the server processing information 124 on the screen 35. For example, the processor 82 displays a bounding box BB based on the first recognition result 122A, in a superimposed state on the medical image 40 displayed in the first display region 35A. The bounding box BB is a rectangular frame-shaped mark for specifying the position of the lesion 42 in the medical image 40. Further, the processor 82 displays text 44A in the second display region 35B. Examples of the text 44A include text for specifying that the lesion 42 is shown in the medical image 40 displayed in the first display region 35A.

In addition, in a case in which the recognition processing 116 executed by the recognition unit 100B is the second recognition processing 116B, the processor 82 displays information based on the second recognition result 122B included in the server processing information 124 on the screen 35. For example, the processor 82 displays the bounding box BB (in the example shown in FIG. 6, the bounding box BB for specifying the position of the lesion 42, which is the processing target of the second recognition processing 116B, in the medical image 40) based on the second recognition result 122B, in a superimposed state on the medical image 40 displayed in the first display region 35A. Further, the processor 82 displays texts 44B, 44C, and 44D in the second display region 35B.

Examples of the text 44B include text for specifying the type of the lesion 42 shown in the medical image 40 displayed in the first display region 35A. Examples of the text 44C include text for specifying the subtype of the lesion 42 shown in the medical image 40 displayed in the first display region 35A. Examples of the text 44D include text for specifying the size of the lesion 42 shown in the medical image 40 displayed in the first display region 35A. Here, the texts 44A to 44D are shown as one of the auxiliary information 44 displayed in the second display region 35B, but visible information (for example, an image) other than the text may also be displayed in the second display region 35B, or the information represented by the texts 44A to 44D may be output as voice.

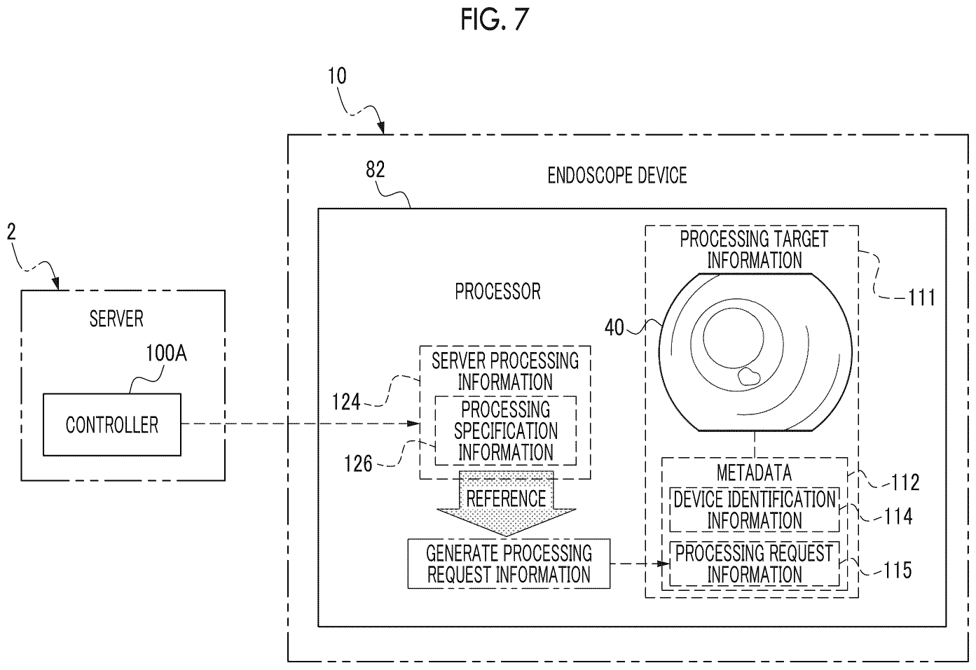

FIG. 7 is a conceptual diagram showing an example of the processing contents executed by the endoscope device 10. As shown in FIG. 7, in a case in which the processing target information 111 is newly generated, the processor 82 generates the processing request information 115 included in the metadata 112 of the processing target information 111 by referring to the processing specification information 126 included in the server processing information 124 acquired from the controller 100A of the server 2. For example, in a case in which it is specified that the first recognition processing 116A or the second recognition processing 116B is executed by the recognition unit 100B from the processing specification information 126 included in the server processing information 124, the processor 82 generates, as the processing request information 115, information for requesting the recognition unit 100B to execute the second recognition processing 116B and includes the generated information in the metadata 112 of the processing target information 111.

It should be noted that, here, although the form example is described in which the processor 82 generates, as the processing request information 115, the information for requesting the recognition unit 100B to execute the second recognition processing 116B in a case in which it is specified that the first recognition processing 116A or the second recognition processing 116B is executed by the recognition unit 100B from the processing specification information 126, this is merely an example. For example, on the premise that the information for requesting the recognition unit 100B to execute the first recognition processing 116A is generated by the processor 82 as the processing request information 115, the processing request information 115 may be changed by the processor 82 from the information for requesting the recognition unit 100B to execute the first recognition processing 116A to the information for requesting the recognition unit 100B to execute the second recognition processing 116B, in accordance with the processing specification information 126. For example, in this case, first, the processor 82 unconditionally generates the information for requesting the recognition unit 100B to execute the first recognition processing 116A as the processing request information 115 each time the medical image 40 is obtained. Then, in a case in which it is specified that the first recognition processing 116A or the second recognition processing 116B is executed by the recognition unit 100B from the processing specification information 126 included in the server processing information 124 transmitted from the server 2, the processor 82 changes the processing request information 115 from the information for requesting the recognition unit 100B to execute the first recognition processing 116A to the information for requesting the recognition unit 100B to execute the second recognition processing 116B.

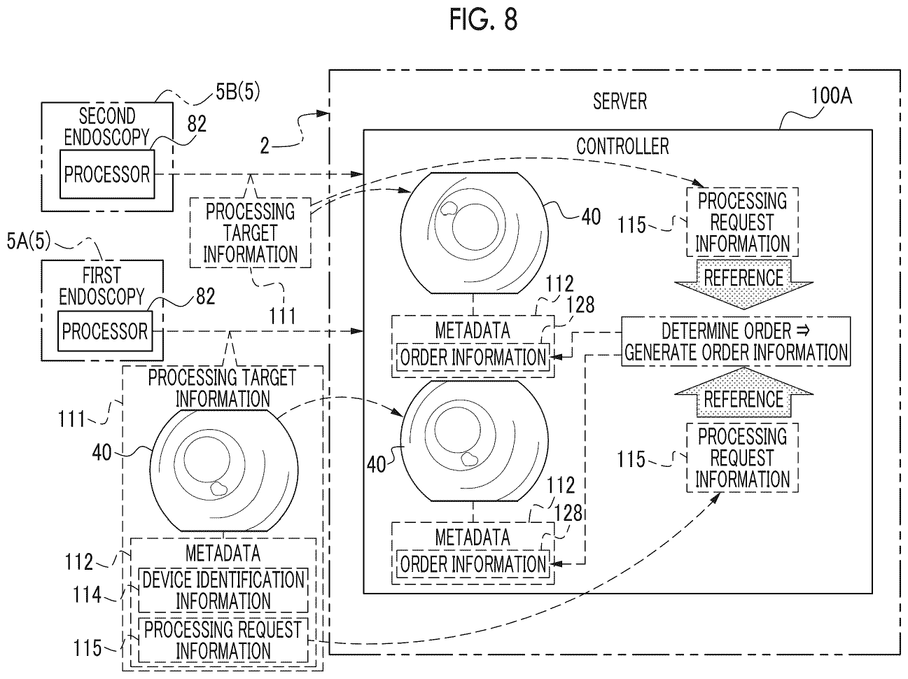

FIG. 8 is a conceptual diagram showing an example of the processing content executed by the server 2 in a case in which the processing target information 111 is transmitted from each of the plurality of endoscope devices 10 to the server 2. As shown in FIG. 8, in a case in which the processing target information 111 is transmitted to the server 2 from the processor 82 of the endoscope device 10 used for the first endoscopy 5A and from the processor 82 of the endoscope device 10 used for the second endoscopy 5B, in the server 2, the controller 100A acquires the processing target information 111 transmitted from the processor 82 of each of the plurality of endoscope devices 10 (here, as an example, the endoscope device 10 used for the first endoscopy 5A and the endoscope device 10 used for the second endoscopy 5B) via the external I/F 98 (FIG. 4).

The controller 100A determines, for each medical image 40, an order in which the recognition processing 116 is executed on the plurality of medical images 40 (in the example shown in FIG. 8, two medical images 40) obtained from each of the plurality of endoscopies 5 (in this case, as an example, the first endoscopy 5A and the second endoscopy 5B), by referring to the processing request information 115 included in the processing target information 111 transmitted from the processor 82 of each of the plurality of endoscope devices 10. The controller 100A generates order information 128 indicating the order determined for each medical image 40 and includes the generated order information in the metadata 112 associated with the corresponding medical image 40.

Here, the order in which the recognition processing 116 is executed refers to an order in which the recognition processing 116 is executed by the recognition unit 100B on the medical image 40 included in the processing target information 111 transmitted from the processor 82 of the endoscope device 10 used for the first endoscopy 5A and on the medical image 40 included in the processing target information 111 transmitted from the processor 82 of the endoscope device 10 used for the second endoscopy 5B.

For example, the first recognition processing 116A (that is, the processing of executing the screening) is given an earlier order than the second recognition processing 116B (that is, the processing of executing the discrimination). The reason for this order is that it is necessary to prioritize the first recognition processing 116A over the second recognition processing 116B in order to prevent the overlooking of the lesion 42 (in other words, the detection failure of the lesion 42) and that a discrimination result of the lesion 42 is less likely to change than a screening result. The reason for prioritizing the first recognition processing 116A over the second recognition processing 116B in order to prevent the overlooking of the lesion 42 is that the lesion 42 is more likely to be erroneously recognized in the screening than in the discrimination in a case in which the lesion 42 is framed out, moves, or is too small and that the overlooking of the lesion 42 can be prevented by executing the AI processing first on the side that is more likely to be erroneously recognized.

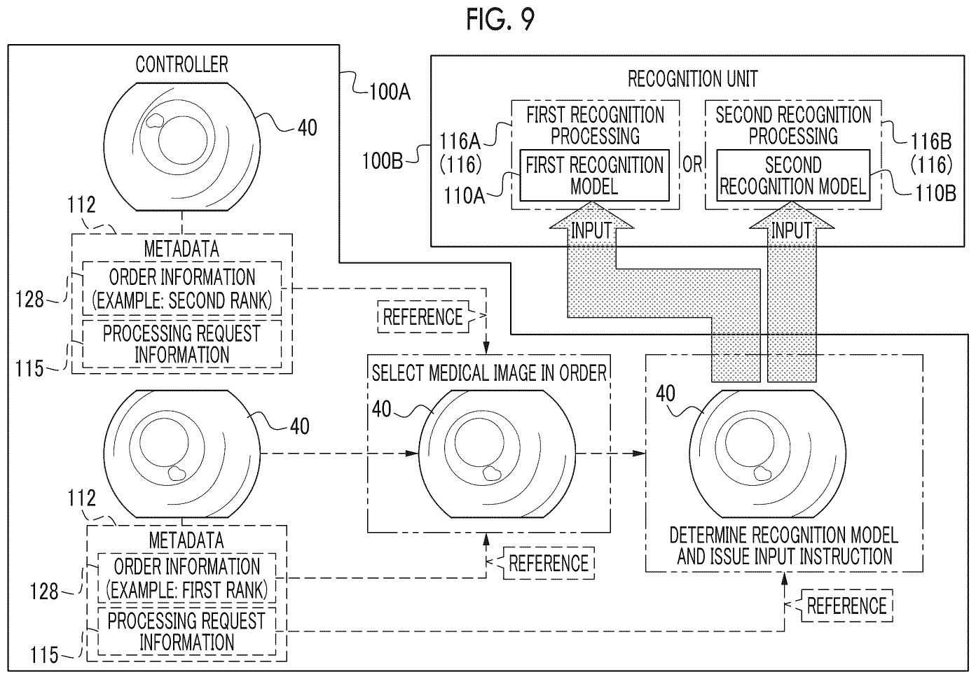

FIG. 9 is a conceptual diagram showing an example of the processing content in the server 2 using the processing request information 115 and the order information 128 included in the metadata 112. As shown in FIG. 9, the controller 100A acquires the order information 128 from the metadata 112 associated with each of the plurality of medical images 40. Then, the controller 100A causes the recognition unit 100B to execute the recognition processing 116 on each of the plurality of medical images 40 in the order (that is, the order determined based on a plurality of pieces of processing request information 115) indicated by the order information 128 acquired from each metadata 112. Hereinafter, a more specific example will be described.

The controller 100A selects the medical image 40 that is the processing target of the recognition processing 116 from among the plurality of medical images 40 in the order indicated by the order information 128. The controller 100A outputs the medical image 40 selected from among the plurality of medical images 40 to the recognition unit 100B. In addition, the controller 100A determines the first recognition model 110A or the second recognition model 110B as the input destination for the medical image 40 selected from the plurality of medical images 40 by referring to the processing request information 115 in the metadata 112 associated with the medical image 40 selected from among the plurality of medical images 40, and designates the determined input destination on the recognition unit 100B.

The recognition unit 100B inputs the medical image 40, which is input from the controller 100A, to the first recognition model 110A or the second recognition model 110B as the input destination designated by the controller 100A. In a case in which designated as the input destination for the medical image 40 by the controller 100A is the first recognition model 110A (that is, in a case in which the medical image 40 is input to the first recognition model 110A), the first recognition result 122A is generated and output by the first recognition model 110A (see FIG. 5). On the other hand, in a case in which designated as the input destination for the medical image 40 by the controller 100A is the second recognition model 110B (that is, in a case in which the medical image 40 is input to the second recognition model 110B), the second recognition result 122B is generated and output by the second recognition model 110B (see FIG. 5).

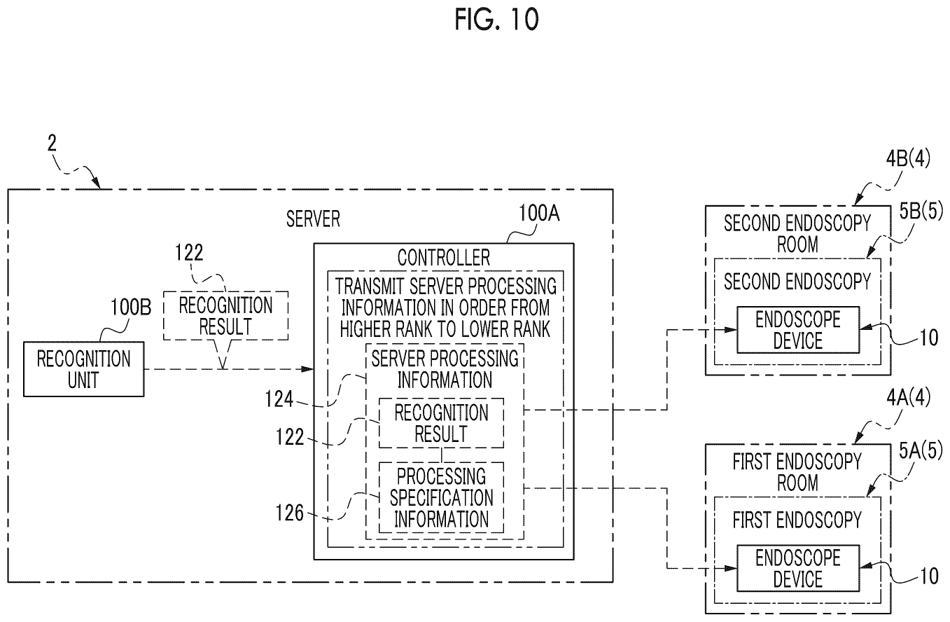

FIG. 10 is a conceptual diagram showing an example of the transmission of the server processing information 124 including the recognition result 122 obtained by executing the recognition processing 116 in the order indicated by the order information 128. As shown in FIG. 10, the controller 100A transmits the server processing information 124 including the recognition result 122 obtained by executing the recognition processing 116 on the medical images 40 selected from a higher rank to a lower rank from among the plurality of medical images 40 in the order indicated by the order information 128, to the endoscope device 10 that is a request source for the execution of the recognition processing 116 in an order from the higher rank to the lower rank (that is, an order in which the recognition results 122 included in the server processing information 124 are obtained). For example, in a case in which the order indicated by the order information 128 in the metadata 112 of the medical image 40 transmitted from the endoscope device 10 used for the first endoscopy 5A is earlier than the order indicated by the order information 128 in the metadata 112 of the medical image 40 transmitted from the endoscope device 10 used for the second endoscopy 5B, the server processing information 124 is transmitted to the endoscope device 10 used for the first endoscopy 5A earlier than to the endoscope device 10 used for the second endoscopy 5B. That is, the server 2 transmits the server processing information 124 including the recognition result 122 for the medical image 40 on which the recognition processing 116 is executed first, to the endoscope device 10 that is the request source for the execution of the recognition processing 116, earlier than the server processing information 124 including the recognition result 122 for the medical image 40 on which the recognition processing 116 is executed later.

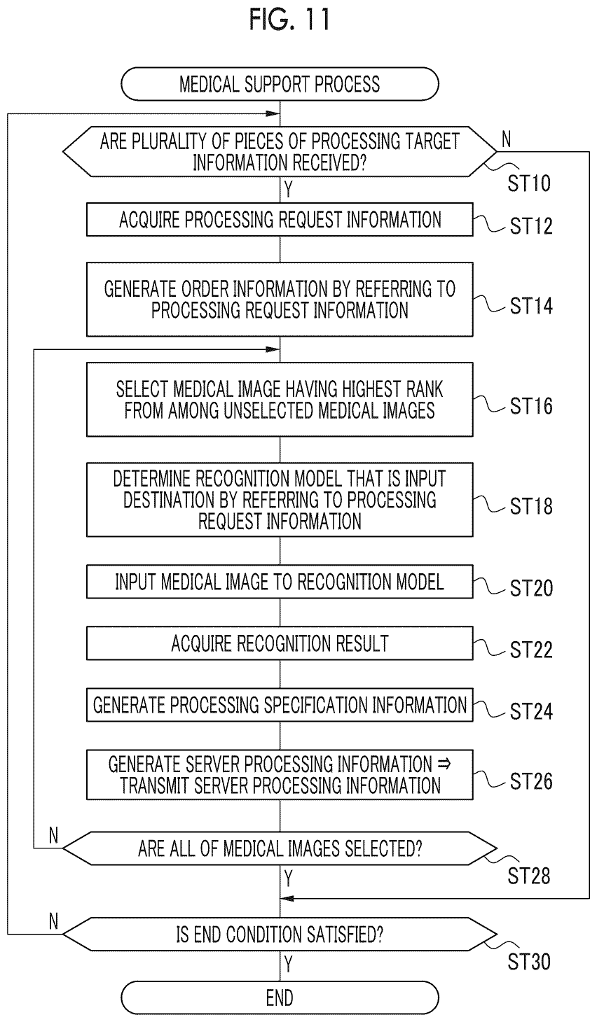

Next, an operation of a part of the medical support system 1 according to the embodiment of the present disclosure will be described with reference to FIG. 11. A flow of the medical support process shown in FIG. 11 is an example of a “medical support method” according to the present disclosure.

In the medical support process shown in FIG. 11, first, in step ST10, the controller 100A determines whether or not a plurality of pieces of processing target information 111 transmitted from the plurality of endoscope devices 10 are received by the external I/F 98. In step ST10, in a case in which the plurality of pieces of processing target information 111 transmitted from the plurality of endoscope devices 10 are not received by the external I/F 98, a negative determination is made, and the medical support process proceeds to step ST30. In step ST10, in a case in which the plurality of pieces of processing target information 111 transmitted from the plurality of endoscope devices 10 are received by the external I/F 98, an affirmative determination is made, and the medical support process proceeds to step ST12.

In step ST12, the controller 100A acquires the processing request information 115 from each of the plurality of pieces of processing target information 111 received in step ST10. After the processing in step ST12 is executed, the medical support process proceeds to step ST14.

In step ST14, the controller 100A generates the order information 128 by referring to the processing request information 115 acquired from each of the plurality of pieces of processing target information 111 received in step ST10. Then, the controller 100A includes the generated order information 128 in the metadata 112 included in each of the plurality of pieces of processing target information 111 received in step ST10 (that is, associates the order information 128 with each medical image 40 included in each of the plurality of pieces of processing target information 111 received in step ST10). After the processing in step ST14 is executed, the medical support process proceeds to step ST16.

In step ST16, the controller 100A selects the medical image 40 of the first order (in other words, the highest rank) in the order indicated by the order information 128 in the metadata 112 from among the medical images 40 that are not selected in step ST16 among the plurality of medical images 40 included in the plurality of pieces of processing target information 111. After the processing in step ST16 is executed, the medical support process proceeds to step ST18.

In step ST18, the controller 100A determines the first recognition model 110A or the second recognition model 110B as the input destination for the medical image 40 selected in step ST16 by referring to the processing request information 115 in the metadata 112 included in the plurality of pieces of processing target information 111. After the processing in step ST18 is executed, the medical support process proceeds to step ST20.

In step ST20, the recognition unit 100B inputs the medical image 40 selected in step ST16 to the first recognition model 110A or the second recognition model 110B determined in step ST18. After the processing in step ST20 is executed, the medical support process proceeds to step ST22.

By executing the processing in step ST20, the recognition result 122 is generated by the first recognition model 110A or the second recognition model 110B determined in step ST18. In step ST22, the controller 100A acquires the recognition result 122. After the processing in step ST22 is executed, the medical support process proceeds to step ST24.

In step ST24, the controller 100A generates the processing specification information 126 as the information for specifying the type of the recognition processing 116 corresponding to the recognition result 122 acquired in step ST22. After the processing in step ST24 is executed, the medical support process proceeds to step ST26.

In step ST26, the controller 100A generates the server processing information 124 including the recognition result 122 acquired in step ST22 and the processing specification information 126 generated in step ST24, and transmits the server processing information 124 having higher priority, in order, to the endoscope device 10 that is the request source for the execution of the recognition processing 116. It should be noted that the priority corresponds to the order indicated by the order information 128 given to the medical image 40 used in order to obtain the recognition result 122 included in the server processing information 124. After the processing in step ST26 is executed, the medical support process proceeds to step ST28.

In step ST28, the controller 100A determines whether or not all of the medical images 40 included in the plurality of pieces of processing target information 111 received in step ST10 are selected in step ST16. In step ST28, in a case in which all of the medical images 40 included in the plurality of pieces of processing target information 111 received in step ST10 are not selected in step ST16, a negative determination is made, and the medical support process proceeds to step ST16. In step ST28, in a case in which all of the medical images 40 included in the plurality of pieces of processing target information 111 received in step ST10 are selected in step ST16, an affirmative determination is made, and the medical support process proceeds to step ST30.

In step ST30, the controller 100A determines whether or not a medical support process end condition is satisfied. Examples of the medical support process end condition include a condition in which an instruction to end the medical support process is given to the medical support system 1 (for example, a condition in which the instruction to end the medical support process is received by the reception device 64 of at least one endoscope device 10 among the plurality of endoscope devices 10).

In a case in which the medical support process end condition is not satisfied in step ST30, a negative determination is made, and the medical support process proceeds to step ST10. In a case in which the medical support process end condition is satisfied in step ST30, an affirmative determination is made, and the medical support process ends.

As described above, in the medical support system 1, the plurality of pieces of processing target information 111 including the plurality of medical images 40 corresponding to the plurality of endoscopies 5 (that is, the plurality of pieces of processing target information 111 each including different medical images 40) are acquired by the controller 100A. Then, the recognition processing 116 is executed on each of the plurality of medical images 40 by the recognition unit 100B in the order determined based on the plurality of pieces of processing request information 115 included in a plurality of metadata 112 corresponding to the plurality of medical images 40 included in the plurality of pieces of processing target information 111.

Therefore, it is possible to suppress a problem (for example, a problem due to a communication time lag between the server 2 and the endoscope device 10) that occurs in a case in which the recognition processing 116 is executed on each of the plurality of medical images 40 corresponding to the plurality of endoscopies 5, as compared to a case in which the recognition processing 116 is executed in parallel on all of the plurality of medical images 40 corresponding to the plurality of endoscopies 5.

In addition, the server 2 transmits the server processing information 124 including the recognition result 122 obtained by executing the recognition processing 116 to the endoscope device 10 that is the request source for the execution of the recognition processing 116. Therefore, the recognition result 122 can be understood by the doctor 12 who is performing the endoscopy 5.

In addition, in the server 2, the server processing information 124 corresponding to the recognition result 122 (in the example shown in FIG. 10, the server processing information 124 including the recognition result 122 and the processing specification information 126) is transmitted to the endoscope device 10 that is the request source for the execution of the recognition processing 116, in the order in which the recognition results 122 are obtained by executing the recognition processing 116 on the plurality of medical images 40 in the order determined based on the processing request information 115 included in the metadata 112 of each of the plurality of pieces of processing target information 111 (that is, the order indicated by the order information 128). Therefore, the recognition result 122 can be understood by the doctor 12 who is performing the endoscopy 5 having higher priority before the doctor 12 who is performing the endoscopy 5 having lower priority.

It should be noted that, in the above-described embodiment, the form example is described in which the metadata 112 includes the processing request information 115, but this is merely an example. For example, processing stage information that is information indicating a stage of the processing executed by the recognition unit 100B may be included in the metadata 112 instead of the processing request information 115. In the processing stage information, the stages of the processing executed by the recognition unit 100B are classified into a stage during the execution of the first recognition processing 116A, a stage after the execution of the first recognition processing 116A and before or during the execution of the second recognition processing 116B, and a stage after the execution of the second recognition processing 116B. In a case in which the processing stage information is used, for example, the controller 100A of the server 2 includes the processing stage information in the server processing information 124 and transmits the server processing information 124 including the processing stage information to the endoscope device 10, and the endoscope device 10 includes, in the metadata 112, the processing stage information included in the server processing information 124 transmitted from the server 2. Then, in a case in which the processing target information 111 including the processing stage information in the metadata 112 is transmitted from the plurality of endoscope devices 10 to the server 2, the controller 100A of the server 2 acquires the processing stage information from the metadata 112 in the processing target information 111 and determines the order by referring to the processing stage information. The stage during the execution of the first recognition processing 116A has an earlier order than the stage after the execution of the first recognition processing 116A and during the execution of the second recognition processing 116B, and the stage after the execution of the first recognition processing 116A and before or during the execution of the second recognition processing 116B has an earlier order than the stage after the execution of the second recognition processing 116B.

The processor 82 of the endoscope device 10 may update the processing request information 115 in accordance with the processing stage information included in the server processing information 124 transmitted from the server 2. For example, information for requesting the recognition unit 100B to execute the second recognition processing 116B need only be used as the processing request information 115 in a case of the stage during the execution of the first recognition processing 116A and in a case of the stage after the execution of the first recognition processing 116A and before or during the execution of the second recognition processing 116B, and information for requesting the recognition unit 100B to execute the first recognition processing 116A is used as the processing request information 115 in a case of the stage after the execution of the second recognition processing 116B.

In the server 2, a part that is the observation target in the endoscopy 5 (for example, an ascending colon, a transverse colon, a descending colon, a sigmoid colon, an upper rectum, and a lower rectum) may be recognized by the recognition unit 100B, and the order may be determined in accordance with the recognized part. For example, in a case in which a part medically known as a part in which a specific lesion such as an SSL is likely to occur is recognized by the recognition unit 100B, the controller 100A determines the order based on the content of the processing request information 115 and the recognized part. In this case, for example, the processing request information 115 need only be given different weights depending on the content, and a plurality of parts need only be given different weights from each other, and the order need only be determined in accordance with a total value of the weights (for example, the larger the total value of the weights is, the earlier the order is).

In addition, the controller 100A may increase a tendency in which the order in a case in which the recognition processing 116 is executed by a WLI method or an LCI method is earlier than the order in a case in which the recognition processing 116 is executed by a BLI method, or vice versa. In this case as well, the weight need only be used in the above-described manner.

In addition, the order may be determined in accordance with the number of the lesions 42 recognized by the recognition unit 100B. For example, the larger the number of the lesions 42 recognized by the recognition unit 100B is, the earlier the controller 100A makes the order. In this case as well, the weight need only be used in the above-described manner.

In addition, in a case in which the recognition unit 100B recognizes a degree of malignancy of the lesion 42, the order may be determined in accordance with the degree of malignancy of the lesion 42. For example, the higher the degree of malignancy of the lesion 42 is, the earlier the controller 100A makes the order. In this case as well, the weight need only be used in the above-described manner.

The controller 100A may set the processing speed in a case in which the recognition processing 116 is executed by the BLI method to be different from the processing speed in a case in which the recognition processing 116 is executed by the WLI method or the LCI method. For example, the server 2 may set the processing speed in a case in which the recognition processing 116 is executed by the BLI method to be higher than the processing speed in a case in which the recognition processing 116 is executed by the WLI method or the LCI method, or vice versa.

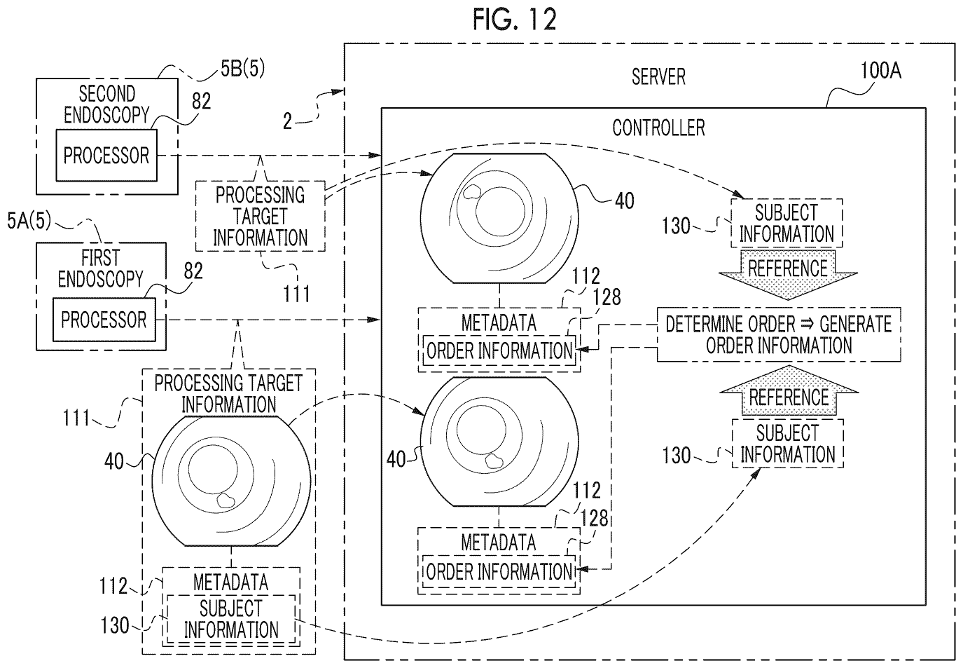

In the above-described embodiment, the form example is described in which the controller 100A determines the order in which the recognition processing 116 is executed by referring to the processing request information 115 included in the processing target information 111 transmitted from the processor 82 of each of the plurality of endoscope devices 10, but this is merely an example, and the present disclosure is not limited thereto. For example, as shown in FIG. 12, the metadata 112 in the processing target information 111 may include subject information 130 that is information on the subject 26 who undergoes the endoscopy 5, and, in this case, the controller 100A may determine the order in which the recognition processing 116 is executed by referring to the subject information 130 included in the metadata 112.

Examples of the subject information 130 include information indicating a medical history of the subject 26, information indicating an age of the subject 26, information indicating a health condition of the subject 26 (for example, information indicating a blood pressure and/or a heart rate), and/or information indicating a gender of the subject 26. It should be noted that the subject information 130 includes middle-of-examination information (here, as an example, information indicating the medical history of the subject 26 and information indicating the gender of the subject 26) that is clarified within a period during which the endoscopy 5 is performed, and pre-examination information (here, as an example, information indicating the health condition of the subject 26) that is clarified before a period during which the endoscopy 5 is performed.

The controller 100A need only derive the priority from the subject information 130 by using a first arithmetic expression (not shown) or a first table (not shown) for deriving the priority from the subject information 130, and determine the order in which the recognition processing 116 is executed based on the derived priority. The order corresponds to the priority derived from the subject information 130, and the higher the priority derived from the subject information 130 is, the earlier the order in which the recognition processing 116 is executed is. For example, the order in which the recognition processing 116 is executed is determined by the controller 100A such that the order in which the recognition processing 116 is executed on the medical image 40 obtained in the endoscopy 5 undergone by the subject 26 having a poor health condition is earlier than the order in which the recognition processing 116 is executed on the medical image 40 obtained in the endoscopy 5 undergone by the subject 26 having a good health condition. In the example shown in FIG. 12, the subject information 130 is an example of “subject information” according to the present disclosure.

In this way, it is possible to suppress a problem that occurs in a case in which the recognition processing 116 is executed on each of the plurality of medical images 40 corresponding to the plurality of endoscopies 5 as compared to a case in which the recognition processing 116 is executed on each of the plurality of medical images 40 corresponding to the plurality of endoscopies 5 in an order determined without considering the information on the subject 26 who undergoes the endoscopy 5.

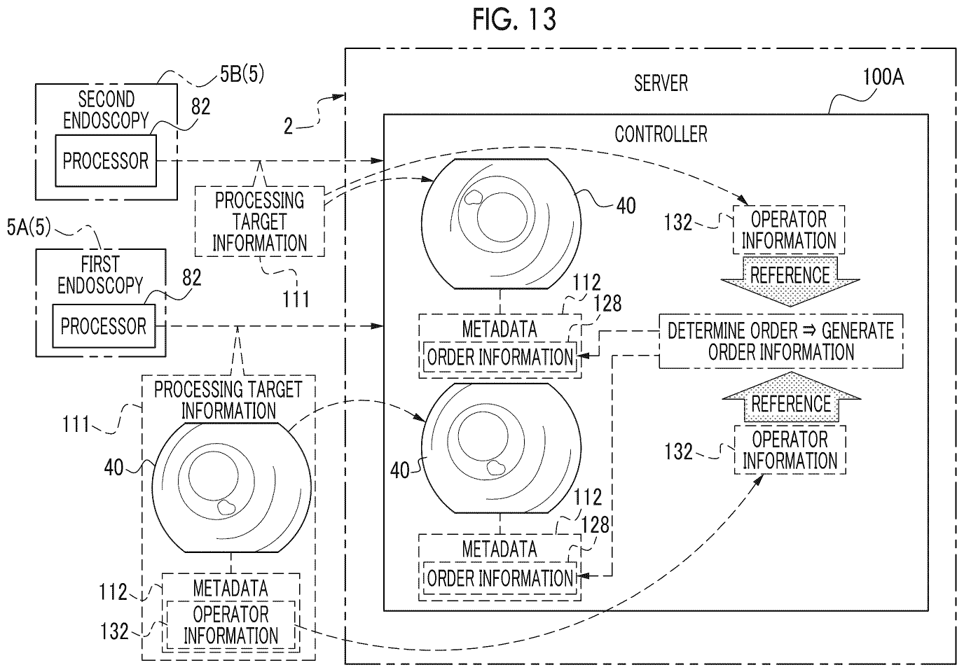

In the example shown in FIG. 12, the form example is described in which the controller 100A determines the order in which the recognition processing 116 is executed by referring to the subject information 130 included in the processing target information 111 transmitted from the processor 82 of each of the plurality of endoscope devices 10, but this is merely an example, and the present disclosure is not limited thereto. For example, as shown in FIG. 13, the metadata 112 in the processing target information 111 may include operator information 132 that is information on the doctor 12 as an operator who performs the endoscopy 5, and, in this case, the controller 100A may determine the order in which the recognition processing 116 is executed by referring to the operator information 132 included in the metadata 112. It should be noted that the operator information 132 is a type of the pre-examination information that is clarified before the period during which the endoscopy 5 is performed.

Examples of the operator information 132 include an identifier for specifying the individual doctor 12, information indicating the total number of years of experience of the doctor 12 in performing endoscopies 5, information indicating a period during which the doctor 12 has not performed endoscopies 5 (that is, a blank period), information indicating the number of times endoscopies 5 have been performed by the doctor 12 within a unit time (for example, within 24 hours), and/or information indicating the field of expertise of the doctor 12.

The controller 100A need only derive the priority from the operator information 132 by using a second arithmetic expression (not shown) or a second table (not shown) for deriving the priority from the operator information 132, and determine the order in which the recognition processing 116 is executed based on the derived priority. The order corresponds to the priority derived from the operator information 132, and the higher the priority derived from the operator information 132 is, the earlier the order in which the recognition processing 116 is executed is. For example, the order in which the recognition processing 116 is executed is determined by the controller 100A such that the order in which the recognition processing 116 is executed on the medical image 40 obtained in the endoscopy 5 performed by the doctor 12 having little practical experience is earlier than the order in which the recognition processing 116 is executed on the medical image 40 obtained in the endoscopy 5 performed by the doctor 12 having a lot of practical experience. In the example shown in FIG. 13, the operator information 132 is an example of “operator information” and “pre-examination information” according to the present disclosure.

In this way, it is possible to suppress a problem that occurs in a case in which the recognition processing 116 is executed on each of the plurality of medical images 40 corresponding to the plurality of endoscopies 5 as compared to a case in which the recognition processing 116 is executed on each of the plurality of medical images 40 corresponding to the plurality of endoscopies 5 in an order determined without considering the information on the doctor 12 as the operator who performs the endoscopy 5.

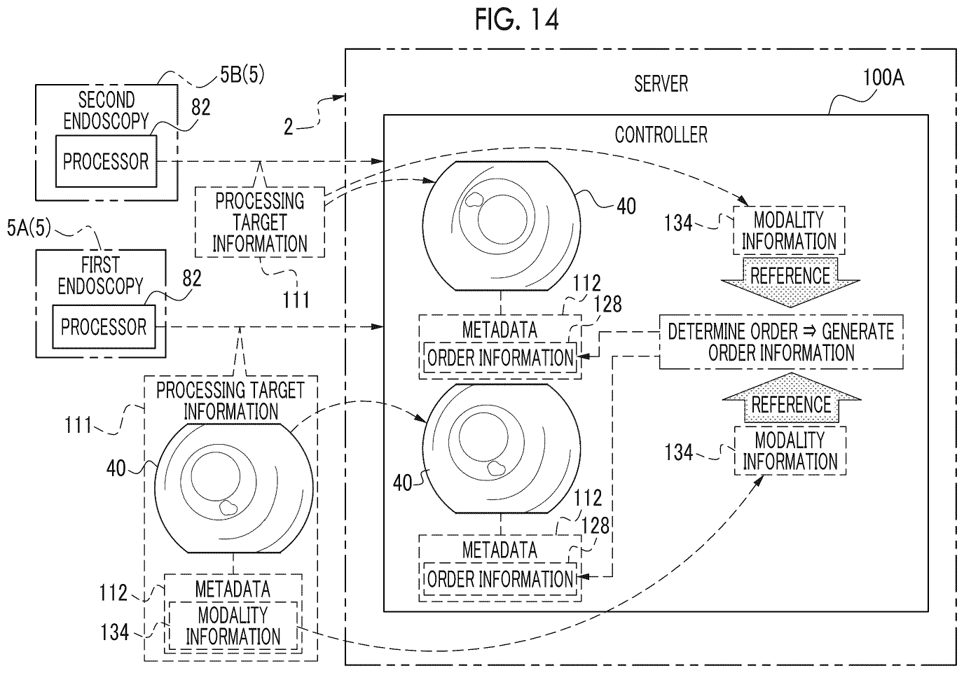

In the example shown in FIG. 13, the form example is described in which the controller 100A determines the order in which the recognition processing 116 is executed by referring to the operator information 132 included in the processing target information 111 transmitted from the processor 82 of each of the plurality of endoscope devices 10, but this is merely an example, and the present disclosure is not limited thereto. For example, as shown in FIG. 14, the metadata 112 in the processing target information 111 may include modality information 134 that is information on the endoscope device 10 as a modality used for the imaging to obtain the medical image 40 in the endoscopy, and, in this case, the controller 100A may determine the order in which the recognition processing 116 is executed by referring to the modality information 134 included in the metadata 112. It should be noted that the modality information 134 is a type of the pre-examination information that is clarified before the period during which the endoscopy 5 is performed.

Examples of the modality information 134 include information indicating the model number of the endoscope device 10, information indicating a time during which the endoscope device 10 is continuously used in the endoscopy 5, information indicating a total use period of the endoscope device 10, and/or information indicating the address for specifying the location of the endoscope device 10 on the network 3.

The controller 100A need only derive the priority from the modality information 134 by using a third arithmetic expression (not shown) or a third table (not shown) for deriving the priority from the modality information 134, and determine the order in which the recognition processing 116 is executed based on the derived priority. The order corresponds to the priority derived from the modality information 134, and the higher the priority derived from the modality information 134 is, the earlier the order in which the recognition processing 116 is executed is. For example, the order in which the recognition processing 116 is executed is determined by the controller 100A such that the order in which the recognition processing 116 is executed on the medical image 40 obtained by performing the imaging via an old endoscope device 10 is earlier than the order in which the recognition processing 116 is executed on the medical image 40 obtained by performing the imaging via a new endoscope device 10. In the example shown in FIG. 14, the modality information 134 is an example of “operator information” and “pre-examination information” according to the present disclosure.

In this way, it is possible to suppress a problem that occurs in a case in which the recognition processing 116 is executed on each of the plurality of medical images 40 corresponding to the plurality of endoscopies 5 as compared to a case in which the recognition processing 116 is executed on each of the plurality of medical images 40 corresponding to the plurality of endoscopies 5 in an order determined without considering the information on the modality used for the imaging to obtain the medical image 40 in the endoscopy 5.

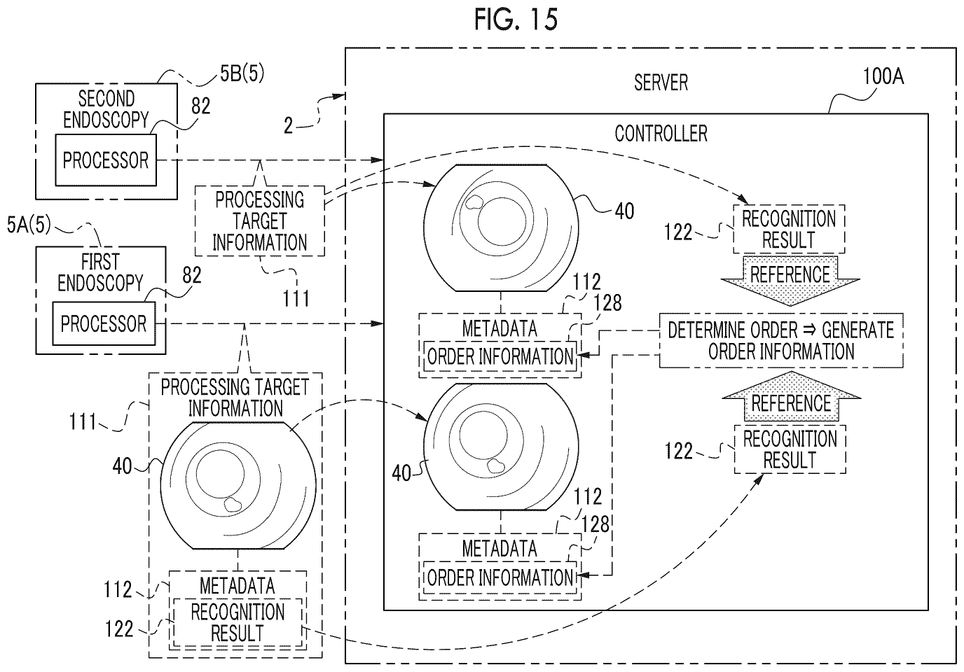

In the example shown in FIG. 14, the form example is described in which the controller 100A determines the order in which the recognition processing 116 is executed by referring to the modality information 134 included in the processing target information 111 transmitted from the processor 82 of each of the plurality of endoscope devices 10, but this is merely an example, and the present disclosure is not limited thereto. For example, as shown in FIG. 15, the metadata 112 in the processing target information 111 may include the recognition result 122 as an intermediate result of the endoscopy 5, and, in this case, the controller 100A may determine the order in which the recognition processing 116 is executed by referring to the recognition result 122 included in the metadata 112. The intermediate result of the endoscopy 5 refers to, for example, information on the lesion 42 shown in the medical image 40 obtained within the period during which the endoscopy 5 is performed. In addition, the intermediate result of the endoscopy 5 can also be referred to as the middle-of-examination information clarified within the period during which the endoscopy 5 is performed. Examples of the information on the lesion 42 shown in the medical image 40 include the screening result and the discrimination result. The recognition result 122 is information specified based on the medical image 40 (that is, information specified by executing the recognition processing 116 on the medical image 40).

The controller 100A need only derive the priority from the recognition result 122 by using a fourth arithmetic expression (not shown) or a fourth table (not shown) for deriving the priority from the recognition result 122, and determine the order in which the recognition processing 116 is executed based on the derived priority. The order corresponds to the priority derived from the recognition result 122, and the higher the priority derived from the recognition result 122 is, the earlier the order in which the recognition processing 116 is executed is. For example, the order in which the recognition processing 116 is executed is determined by the controller 100A such that the order in which the recognition processing 116 is executed on the medical image 40 associated with the first recognition result 122A (that is, the screening result) is earlier than the order in which the recognition processing 116 is executed on the medical image 40 associated with the second recognition result 122B (that is, the discrimination result). The reason for this is that, in the endoscopy 5, the discrimination result is less likely to change than the screening result and that it is preferable for the doctor 12 to understand the screening result earlier than the discrimination result. It should be noted that this is merely an example, and the order in which the recognition processing 116 is executed may be determined by the controller 100A such that the order in which the recognition processing 116 is executed on the medical image 40 associated with the second recognition result 122B (that is, the discrimination result) is earlier than the order in which the recognition processing 116 is executed on the medical image 40 associated with the first recognition result 122A (that is, the screening result). In the example shown in FIG. 15, the recognition result 122 is an example of an “intermediate result” and “middle-of-examination information” according to the present disclosure. The first recognition result 122A (that is, the screening result) and the second recognition result 122B (that is, the discrimination result) are examples of “feature region information” according to the present disclosure.

In this way, since the order is determined based on the intermediate result of the endoscopy 5, it is possible to suppress a problem that occurs in a case in which the recognition processing 116 is executed on each of the plurality of medical images 40 corresponding to the plurality of endoscopies 5 as compared to a case in which the recognition processing 116 is executed on each of the plurality of medical images 40 corresponding to the plurality of endoscopies 5 in an order determined without considering the intermediate result of the endoscopy 5 (for example, the screening result and the discrimination result that are the information on the lesion 42 shown in the medical image 40 obtained within the period during which the endoscopy 5 is performed).

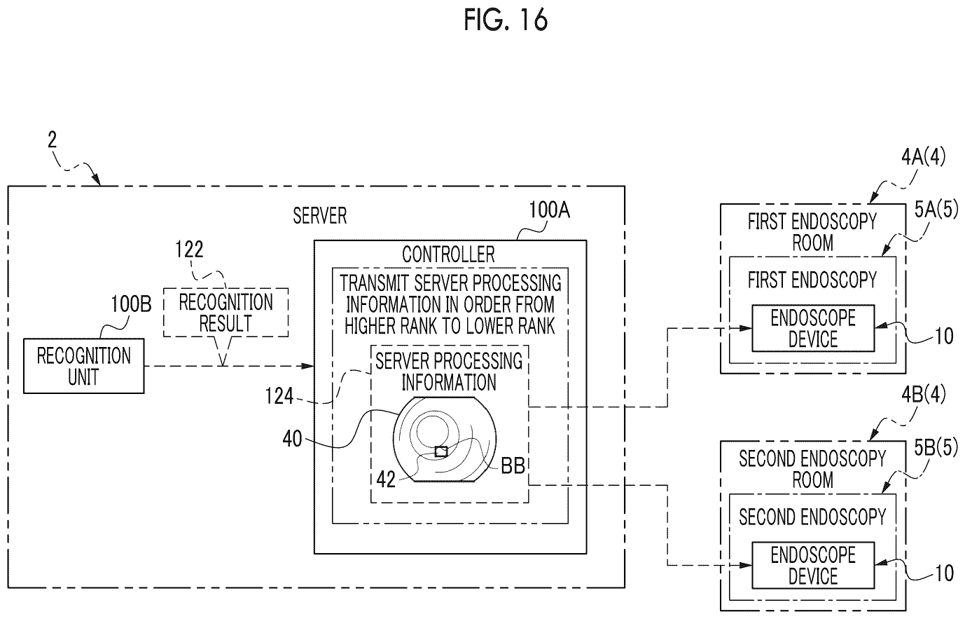

In the above-described embodiment, the server processing information 124 including the recognition result 122 and the processing specification information 126 associated with each other is described as an example, but this is merely an example, and the present disclosure is not limited thereto. For example, information in which the medical image 40 used for the recognition processing 116 and the recognition result 122 are associated with each other may be included in the server processing information 124. For example, in a case in which the recognition result 122 includes the bounding box BB, as shown in FIG. 16, the server processing information 124 may include information in which the bounding box BB as one of the pieces of information included in the recognition result 122 is given to the medical image 40 used for the recognition processing 116. In this way, in the endoscope device 10, the processor 82 (see FIG. 3) can receive the server processing information 124 transmitted from the server 2, acquire the information in which the bounding box BB is given to the medical image 40 used for the recognition processing 116 from the server processing information 124 received from the server 2, and display the acquired information on the screen 35 (for example, the first display region 35A).

It should be noted that, here, the information in which the bounding box BB as one of the pieces of information included in the recognition result 122 is given to the medical image 40 used for the recognition processing 116 is described as an example, but the server processing information 124 may include information in which the screening result and the discrimination result other than the bounding box BB (for example, the texts 44A, 44B, 44C, and 44D shown in FIG. 6, or images to be substituted for the texts 44A, 44B, 44C, and 44D) are associated with the medical image 40.

In the above-described embodiment, the controller 100A determines the order only by referring to the processing request information 115, but this is merely an example. For example, the controller 100A may determine the order by making a comprehensive determination from a plurality of pieces of information among the processing request information 115, the recognition result 122, the subject information 130, the operator information 132, the modality information 134, the processing stage information, the information indicating the part, and the information indicating the degree of malignancy of the lesion 42. In this case, for example, the controller 100A may give different weights to the plurality of pieces of information among the processing request information 115, the recognition result 122, the subject information 130, the operator information 132, the modality information 134, the processing stage information, the information indicating the part, and the information indicating the degree of malignancy of the lesion 42, and determine the order based on the total value of the weights.

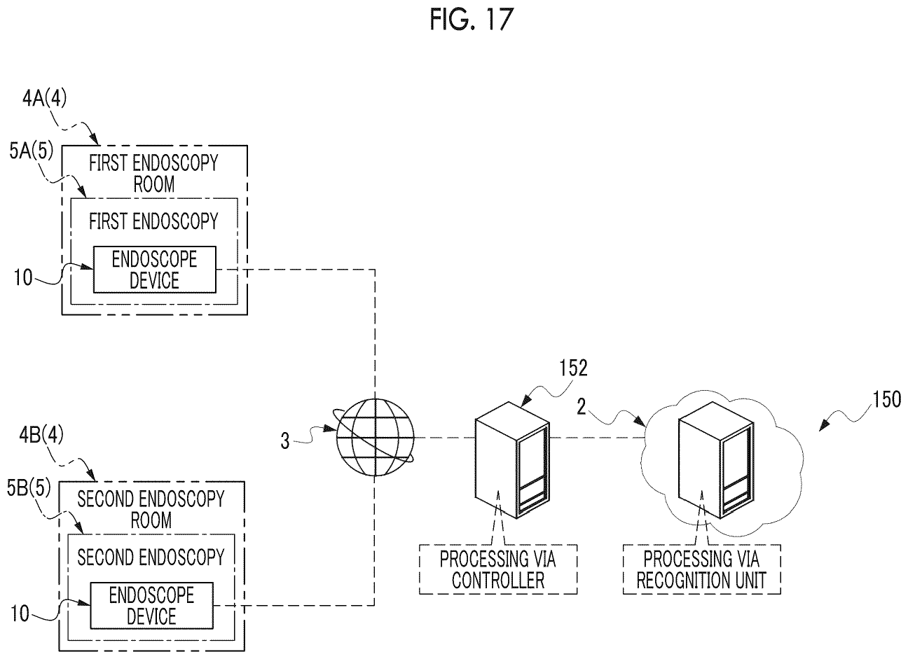

In the above-described embodiment, the form example is described in which the controller 100A of the server 2 causes the recognition unit 100B to execute the recognition processing 116 on each of the plurality of medical images 40 in the order determined based on the information included in the processing target information 111, but this is merely an example. For example, as shown in FIG. 17, a medical support system 150 may realize processing using the controller 100A and processing (that is, the recognition processing 116) using the recognition unit 100B.

The medical support system 150 is different from the medical support system 1 according to the above-described embodiment in that the medical support system 150 includes a management device 152. The management device 152 is connected to the network 3. The server 2 is connected to the network 3 via the management device 152. The management device 152 is a device that manages the transmission and the reception of various types of information between the plurality of endoscope devices 10 and the server 2. The management device 152 executes the processing using the controller 100A according to the above-described embodiment, and the server 2 executes the processing using the recognition unit 100B according to the above-described embodiment. That is, the management device 152 causes the server 2 to execute the recognition processing 116 on each of the plurality of medical images 40 in the order determined based on the information included in the processing target information 111. Then, the management device 152 acquires, from the server 2, the recognition result 122 obtained by executing the recognition processing 116 via the server 2, generates the server processing information 124 in the same manner as in the above-described embodiment, and transmits the generated server processing information 124 to the plurality of endoscope devices 10. The server processing information 124 may be generated by the server 2 instead of by the management device 152. In the example shown in FIG. 17, the management device 152 is an example of a “medical support device” according to the present disclosure, and the server 2 is an example of a “processing device” according to the present disclosure.

In the above-described embodiment, the lesion 42 is described as an example of the “first feature region” and the “second feature region” according to the present disclosure, but the present disclosure is not limited thereto, and the present disclosure is also applicable in a case in which a resection region, a bleeding region, a marking region, an organ, or a treatment tool (for example, a hemostatic clip placed inside the body) is applied instead of the lesion 42.

In the above-described embodiment, the camera 52 mounted in the endoscope 16 is described as an example, but the technology of the present disclosure is not limited thereto. For example, the present disclosure is also applicable to a camera mounted in a fundus camera or in a medical microscope.

In the above-described embodiment, the medical image 40 obtained by imaging the inside of the large intestine 28 via the camera 52 mounted in the endoscope 16 is described as an example, but this is merely an example, and the medical image 40 may be a medical image obtained by various modalities such as a radiography apparatus, MRI, CT, or an ultrasound diagnostic device (for example, an ultrasound endoscope device).

In the above-described embodiment, the recognition processing 116 using AI of the bounding box method is described as an example, but this is merely an example, and, for example, recognition processing using AI of a segmentation method may be executed instead of the recognition processing 116 using AI of the bounding box method. In addition, instead of the recognition processing using the AI method, recognition processing using a non-AI method (for example, a template matching method) may be executed, or recognition processing using a combination of the non-AI method and the AI method may be executed.

In the above-described embodiment, the form example is described in which the medical support process is executed by the computer 96, but the present disclosure is not limited thereto, and at least a part of the processing included in the medical support process may be executed by a device provided outside the computer 96.

In the above-described embodiment, although the form example is described in which the server 2 is realized by cloud computing, this is merely an example. The server 2 may be realized by network computing such as fog computing, edge computing, or grid computing.

In the above-described embodiment, the form example is described in which the medical support program 108 is stored in the storage 104, but the present disclosure is not limited thereto. For example, the medical support program 108 may be stored in a portable computer-readable non-transitory storage medium such as an SSD or a USB memory. The medical support program 108 stored in the non-transitory storage medium is installed in the computer 96 of the endoscope device 10. The processor 100 executes the medical support process in accordance with the medical support program 108.

The medical support program 108 may be stored in a storage device of another computer or server device connected to the endoscope device 10 via a network, and the medical support program 108 may be downloaded in response to a request from the endoscope device 10 and installed in the computer 96.

It should be noted that it is not necessary to store the entire medical support program 108 in a storage device of another computer or server device connected to the endoscope device 10 or to store the entire medical support program 108 in the storage 104, and a part of the medical support program 108 may be stored.

Various processors described below can be used as the hardware resource for executing the medical support process. Examples of the processor include a CPU which is a general-purpose processor that functions as the hardware resource executing the medical support process by executing software, that is, a program. In addition, examples of the processor include a dedicated electric circuit which is a processor of which a circuit configuration is specially designed for executing specific processing, such as an FPGA, a PLD, or an ASIC. Any processor has a memory built in or connected to it, and any processor executes the medical support process by using the memory.

The hardware resource for executing the medical support process may be configured by one of the various processors or by combining two or more processors of the same type or different types (for example, by combining a plurality of FPGAs or by combining a CPU and an FPGA). The hardware resource for executing the medical support process may also be one processor.

As an example of the configuration using one processor, first, there is a form in which one processor is configured by combining one or more CPUs and software, and the processor functions as the hardware resource for executing the medical support process. As a second example, as typified by an SoC or the like, there is a form in which a processor that realizes all functions of a system including a plurality of hardware resources executing the medical support process with one IC chip is used. As described above, the medical support process is realized by using one or more of the various processors as the hardware resource.

As a hardware structure of these various processors, more specifically, an electric circuit in which circuit elements, such as semiconductor elements, are combined can be used. Further, the medical support process is merely an example. Therefore, it goes without saying that unnecessary steps may be deleted, new steps may be added, or the processing order may be changed within a range that does not deviate from the gist.

The above-described contents and the above-shown contents are the detailed description of the parts according to the present disclosure, and are merely examples of the present disclosure. For example, the description of the configuration, the function, the operation, and the effect is the description of examples of the configuration, the function, the operation, and the effect of the parts according to the present disclosure. Accordingly, it goes without saying that unnecessary parts may be deleted, new elements may be added, or replacements may be made with respect to the above-described contents and the above-shown contents within a range that does not deviate from the gist of the present disclosure. In addition, in order to avoid complications and facilitate understanding of the parts according to the present disclosure, the description of common technical knowledge or the like, which does not particularly require the description for enabling the implementation of the present disclosure, is omitted in the above-described contents and the above-shown contents.

All of the documents, the patent applications, and the technical standards described in the present specification are incorporated into the present specification by reference to the same extent as in a case in which the individual documents, patent applications, and technical standards are specifically and individually stated to be described by reference.

### EXPLANATION OF REFERENCES

What is claimed is:

1. A medical support device comprising:
a processor,
wherein the processor
acquires a plurality of medical images corresponding to a plurality of medical examinations,
acquires a plurality of pieces of medical examination information that correspond to the plurality of medical examinations and that are each associated with the corresponding medical examination, and
causes a processing device to execute recognition processing on each of the plurality of medical images in an order determined based on the plurality of pieces of medical examination information.

2. The medical support device according to claim 1,
wherein the medical examination information includes middle-of-examination information clarified within a period during which the corresponding medical examination is performed.

3. The medical support device according to claim 1,
wherein the medical examination information includes pre-examination information clarified before a period during which the corresponding medical examination is performed.

4. The medical support device according to claim 1,
wherein the medical examination information includes subject information on a subject who undergoes the corresponding medical examination.

5. The medical support device according to claim 1,
wherein the medical examination information includes operator information on an operator who performs the corresponding medical examination.

6. The medical support device according to claim 1,
wherein the medical image is obtained in the corresponding medical examination, and
the medical examination information includes modality information on a modality used for imaging to obtain the medical image in the corresponding medical examination.

7. The medical support device according to claim 1,
wherein the medical image is obtained in the corresponding medical examination,
the medical examination information includes an intermediate result of the corresponding medical examination, and
the intermediate result is information specified based on the medical image obtained in the corresponding medical examination.

8. The medical support device according to claim 7,
wherein the intermediate result includes feature region information that is information on a first feature region shown in the medical image.

9. The medical support device according to claim 8,
wherein the first feature region is a lesion, and
the intermediate result is a screening result of the lesion or a discrimination result of the lesion.

10. The medical support device according to claim 1,
wherein the processor transmits medical support information including an execution result obtained by executing the recognition processing to a device used for the medical examination corresponding to the medical image that is a target of the recognition processing executed to obtain the execution result included in the medical support information.

11. The medical support device according to claim 10,
wherein the medical support information includes information in which the medical image that is the target of the recognition processing executed to obtain the execution result included in the medical support information and the execution result are associated with each other.

12. The medical support device according to claim 10,
wherein the processor transmits corresponding medical support information to the device in an order in which the execution result is obtained via the recognition processing executed on each of the plurality of medical images in the order.

13. The medical support device according to claim 1,
wherein the recognition processing is processing of generating information on a second feature region by inputting the medical image to a trained model that receives input of an image showing a first region corresponding to the second feature region to generate information on the first region.

14. The medical support device according to claim 1,
wherein each of the plurality of medical examinations is an endoscopy, and
each of the plurality of medical images is an endoscopic image obtained in the endoscopy.

15. A medical support system comprising:
the medical support device according to claim 1; and
a communication device that transmits the medical image to the medical support device and that receives an execution result obtained by executing the recognition processing.

16. A medical support method comprising:
acquiring a plurality of medical images corresponding to a plurality of medical examinations;
acquiring a plurality of pieces of medical examination information that correspond to the plurality of medical examinations and that are each associated with the corresponding medical examination; and
causing a processing device to execute recognition processing on each of the plurality of medical images in an order determined based on the plurality of pieces of medical examination information.

17. A non-transitory computer-readable storage medium storing a program executable by a computer to execute a process comprising:
acquiring a plurality of medical images corresponding to a plurality of medical examinations;
acquiring a plurality of pieces of medical examination information that correspond to the plurality of medical examinations and that are each associated with the corresponding medical examination; and
causing a processing device to execute recognition processing on each of the plurality of medical images in an order determined based on the plurality of pieces of medical examination information.
